(19) United States
(12) Patent Application Publication
(10) Pub. No.: US 2016/0229780 A1
(43) Pub. Date: Aug. 11, 2016

(54) PROCESS FOR THE PURIFICATION OF ACETONE FROM THE KETONIZATION OF ACETIC ACID

(71) Applicant: Eastman Chemical Company, Kingsprt, TN (US)

(72) Inventors: Scott Donald Barnicki, Kingsport, TN (US); Randy Lynn Jennings, Gate City, VA (US); Daniel McNabb, Kingsport, TN (US); Jerry Lynn Miller, Kingsport, TN (US); James Eric Ward, Gray, TN (US)

(73) Assignee: EASTMAN CHEMICAL COMPANY, Kingsport, TN (US)

(21) Appl. No.: 14/618,466

(22) Filed: Feb. 10, 2015

(51) Int. Cl. C07C 45/84; B01D 3/36

(52) U.S. Cl. CPC C07C 45/84; B01D 3/36

(57) ABSTRACT

A method for purification of acetone including the steps of: a) feeding a crude product mixture comprising acetone, water, an impurity to a distillation column; b) withdrawing a liquid side draw stream from the distillation column; c) separating the side draw stream into a water layer and an organic layer, and returning the water layer to the distillation column; and d) recovering from the distillation column: (i) a lower boiling fraction comprising a purified acetone, relative to the crude product mixture and (ii) a higher boiling fraction comprising a major amount of the water.

### FIELD OF THE INVENTION

This invention pertains to the purification of acetone produced by the ketonization of acetic acid. More particularly, this invention pertains to the purification of acetone using fractional distillation in single distillation column in which ketonization by-products are removed from the distillation in a side draw stream as a water azeotrope.

### BACKGROUND OF THE INVENTION

It is known to those skilled in the art that numerous industrial processes are presently used to manufacture acetone. One process for producing acetone includes dehydrogenation of 2-propanol. Propylene is absorbed in concentrated sulfuric acid to produce isopropyl sulfate, which is then hydrolyzed to 2-propanol. The 2-propanol is then oxidized to produce acetone. It is also known that alumina-supported platinum or rhodium catalysts can be used for dehydrogenating lower secondary alcohols to ketones.

Acetone may also be produced by reacting formaldehyde with methyl chloride to produce acetone and hydrogen chloride. Methyl chloride is a toxic gas however, and formaldehyde is a known carcinogen.

Another method more commonly used is obtaining acetone as a co-product of phenol production where benzene is alkylated in the presence of a catalyst with propylene to produce cumene. Cumene is in turn oxidized to cumene hydroperoxide (CHP), which is then hydrolyzed in an acidic medium to yield phenol and acetone. Crude acetone resulting from the production of phenol from cumene typically contains about 200-700 ppm aldehydes and 200-500 ppm methanol. Traditionally, removal of light aldehyde impurities is accomplished by reactive distillation in which an aqueous solution of sodium hydroxide is injected into the distillation column to promote condensation of aldehydes to form higher-boiling compounds. Treatment of acetone with aqueous sodium hydroxide during distillation leads to production of distillation bottoms containing large amounts of polymers and salts, thereby decreasing the efficiency of conventional reboilers. Moreover, base-catalyzed self-condensation of acetone reduces the yield of purified acetone. Impurity levels in commercial acetone purified by this method are still about 30-50 ppm for acetaldehyde and about 200-300 ppm for methanol.

Acetic acid and acetic anhydride are frequently used as solvents, to prepare acetate esters and to prepare other, high-boiling anhydrides. Some examples of chemical processes that produce an acetyl byproduct stream include, but are not limited to, acetylation of wood, acetylation of alcohols with acetic anhydride to form esters, carbonylation of methanol and methyl acetate to form acetic acid and acetic anhydride, preparation of ketenes and diketene from acetic acid; polymerization reactions, such as condensation of phenyl acetate monomers to produce polyesters or polycarbonates; preparation of fine chemicals and pharmaceuticals; preparation of carboxylic acid anhydrides from their corresponding acids by exchange with acetic anhydride; and acylation reactions. Examples for fine chemical and pharmaceutical products include but are not limited, to industrial production of ibuprofen and liquid crystal polymers. Recovery and reuse of acetyl byproduct streams from these applications improves the overall acetyl efficiency, thereby greatly reducing the cost of the acetyl feedstock. A disadvantage of these acetyl byproduct streams is that the byproduct stream frequently contains a complex mixture of impurities that form azeotropes or distillation pinch points with acetic acid and cannot be easily separated without a complex and costly distillation scheme.

As used herein, the term “azeotrope” is intended to have its commonly accepted meaning as would be understood by persons having ordinary skill in the art; that is, a compound, blend or mixture having a constant boiling temperature and having a constant composition which is the same in both vapor and liquid. The relative volatility of the components of an azeotrope at the azeotropic composition is unity. Azeotropes may be determined experimentally or by calculations based on the vapor equilibrium properties of the chemical components. These techniques are well known to persons skilled in the art such as, for example, by using group contribution methods as exemplified by the UNIFAC method. The present invention includes binary and ternary azeotropes containing acetic acid as one of the components.

The term “pinch point” as used herein, is intended to have its commonly understood meaning in the distillation arts, that is, a binary system that exhibits a region of low relative volatility where the y-x vapor-liquid equilibrium line (y=vapor mole fraction, x=liquid mole fraction) tangentially approaches the y=x line. Such systems are commonly referred to as having “pinched” vapor-liquid equilibrium or as “pinched” systems. Separation of component mixtures typically can be accomplished by distillation and are based on differences in vapor and liquid compositions. Since pinched systems show regions with increasingly small differences in vapor-liquid composition, separation by distillation may be difficult, requiring high reflux ratios and/or a large number of theoretical stages to effect any separation.

The term “azeotrope-forming impurity” as used herein means a compound that forms an azeotrope with acetic acid. The azeotrope may be high boiling (known as a maximum boiling azeotrope), wherein the boiling point at the azeotrope composition is greater than the boiling points of the pure components at a constant pressure. The azeotrope also may be low boiling (known as a minimum boiling azeotrope), wherein the boiling point at the azeotrope composition is less than the boiling points of the pure components at a constant pressure. Some examples of various compounds that form minimum-boiling azeotropes with acetic acid include, but are not limited to, aromatic compounds, such as for example, benzene, toluene, xylenes, butyl benzenes, isopropyl toluenes, phenylacetates, styrene, ethylbenzene, and the like; hydrocarbons, such as, for example, heptane, octane, various alkenes and terpenes, such as limonene, a-pinene, β-pinene, camphene, and the like; ketones such as, for example, 4-methyl-2-pentanone, substituted acetophenones; esters, such as phenylacetates; alkyl halides, aryl haldides, and hydroxyalkyl halides such as, for example, epichlorohydrin, 2-iodopropane, 1-iodopropane, 1,2-dichloropropane, 1-bromobutane, 2-bromobutane, isobutyl bromides, iodobutanes, iodoisobutanes, bromobenzene, chlorobenzene, and ethyl dibromide; sulfur-containing species, such as, for example dimethyl sulfoxide, tetrahydrothiophene, diethylsulfide, and diisopropyl sulfide; amines, amides, and other nitrogen-containing species, such as for example, trimethylamine, triethylamine, pyridine, methyl pyridines, dimethyl pyridines, dimethyl acetamide, and nitroethane; alcohols, such as isobutanol, 3-methylbutanol; and esters, such as, for example isobutyl formate, and isobutyl acetate.

For the purpose of discussion of the invention and claims herein, both terms “azeotrope” and “pinch point” will be designated herein as “azeotrope” and/or “azeotrope-forming” due to the difficulty in vapor-liquid distillative separations of such compositions notwithstanding the commonly understood meaning of each.

In the acetylation of wood, acetic anhydride is contacted with wood at high temperatures and pressures. The wood acetylation process produces a byproduct stream containing acetic acid, acetic anhydride, and various terpene and terpenoid impurities that are extracted from the wood during the acetylation reaction. These terpenes and terpenoid compounds form azeotropes with acetic acid.

Similarly, in the preparation of diketene from acetic acid, acetic acid is dehydrated at high temperature to form a ketene which is condensed and absorbed in diketene solvent where it further dimerizes to form diketene. The crude diketene absorbent is then distilled to produce in the overhead a purified diketene. The bottoms product, “diketene sludge,” contains acetic acid, water, acetone, and a host of impurities that form one or more azeotropes with acetic acid.

In another example, isobutyric anhydride is produced by acetyl exchange with acetic anhydride. The isobutyric anhydride is thermally cracked to form dimethylketene, which is then purified and dimerized to give 2,2,4,4-tetramethyl-1,3-butanedione. Dimerization is followed by hydrogenation to 2,2,4,4-tetramethyl-1,3-butanediol. The isobutyric acid recycled from the dimethylketene furnace to the isobutyric anhydride production unit contains a variety of impurities, such as for example, 2,4-dimethyl-1,3-pentadiene, tetramethylethylene, diisopropyl ketone, and isopropyl isopropenyl ketone, which result from the high temperature cracking process. Many of these impurities form azeotropes with acetic acid, and contaminate the acetyl stream from the isobutyric anhydride production unit. These acetic acid azeotrope-forming impurities cannot be separated from acetic acid by simple fractional distillation.

The ketonization of acetic acid with itself and other carboxylic acids, esters, or aldehydes is a valuable and efficient means for the synthesis of acetone and other methyl ketones. Although the ketonization reaction typically gives high yields, it produces water and small amounts of various organic by-products that must be separated from the desired ketone products. For example, the ketonization of acetic acid to acetone coproduces one mole of water for each mole of acetone produced in addition to small amounts of heavier organic compounds, such as mesityl oxide, mesitylene, isophorone, methyl ethyl ketone, methyl propyl ketone, and isopropyl isopropenyl ketone. A crude product stream from ketonization of acetic acid, therefore, will contain acetone, water, and a host of organic impurities. Purification of this crude product stream to give a purified acetone product and a water stream suitable for disposal in compliance with environmental laws will typically result in a purified acetone product, a water stream free of by-product organics, and a stream concentrated in by-product organics. This separation generally will require at least two distillation columns and a decanter and is capital- and energy-intensive. A distillation process, therefore, could greatly improve the efficiency and reduce the equipment and processing costs associated the acetic acid ketonization process.

Accordingly, there is a need for a method by which the acetic acid from such byproduct streams can be utilized or converted to another product with a reduction or elimination of the acetic acid azeotrope-forming compound(s).

### SUMMARY OF THE INVENTION

It has been discovered that an acetic acid by-product stream contaminated with up to about 50 weight percent of at least one impurity that forms an azeotrope or pinch point with acetic acid can be converted to acetone by a ketonization process whereby the azeotrope-forming impurity can be separated from the acetone product by distillation. It has also been discovered that crude acetone product mixture of the ketonization of acetic acid can be efficiently purified using a simplified, one-column distillation process instead of the conventional two-column distillation scheme. Accordingly, the invention is a process for the purification of acetone, comprising: (a) feeding a crude product mixture comprising acetone, water, and an impurity to a distillation column; (b) withdrawing a liquid side draw stream from the distillation column; (c) separating the side draw stream into a water layer and an organic layer, and returning the water layer to the distillation column; and (d) recovering from the distillation column: (i) a lower boiling fraction comprising a purified acetone, relative to the crude product mixture and (ii) a higher boiling fraction comprising a major amount of the water.

The present invention includes a process for preparing a purified ketone from an acetic acid containing stream comprising the steps of: a) contacting the acetic acid containing feed stream comprising acetic acid and an impurity comprising at least one acetic acid azeotrope-forming compound with a meal oxide catalyst in a ketonization reactor to produce a crude product mixture by a ketonization reaction wherein the crude product mixture comprises acetone, water, the impurity, and by-products from the ketonization reaction; b) feeding the crude product mixture comprising acetone, water, and an impurity to a distillation column; c) withdrawing a liquid side draw stream from the distillation column; d) separating the side draw stream into a water layer and an organic layer, and returning the water layer to the distillation column; and e) recovering from the distillation column: (i) a lower boiling fraction comprising a purified acetone, relative to the crude product mixture and (ii) a higher boiling fraction comprising a major amount of the water.

The present invention also includes a process for preparing a purified ketone from an acetic acid containing stream comprising the steps of: a) vaporizing an acetyl feed stream comprising acetic acid, an impurity comprising at least one acetic acid azeotrope-forming compound, and 0-50 weight % water, optionally mixing steam with the vaporized acetyl feed stream to produce a vaporized feed mixture; b) superheating the vaporized feed mixture to produce a superheated feed mixture; c) contacting the superheated feed mixture with a metal oxide catalyst in a ketonization reactor to produce a crude product mixture comprising acetone, water, the impurity, carbon dioxide, and byproducts from the ketonization reaction; d) recovering condensable components of the crude product mixture to produce a recovered liquid crude acetone stream comprising acetone, water, an impurity and ketonization by-products and a gaseous off-gas stream; e) feeding the recovered liquid crude acetone stream to a distillation column; f) withdrawing a liquid side draw stream from the distillation column; g) separating the side draw stream into a water layer and an organic layer, and returning the water layer to the distillation column; and h) recovering from the distillation column: (i) a lower boiling fraction comprising a purified acetone relative to the crude product mixture and (ii) a higher boiling fraction comprising a major amount of the water.

### BRIEF DESCRIPTION OF THE DRAWINGS

FIG. 1 is a schematic diagram depicting possible conversion compounds of acetic acid during ketonization.

FIG. 2 is a block diagram of a ketonization process for converting acetic acid to acetone for feed to a distillation column in accordance with the present invention.

FIG. 3 is a block diagram of the side draw distillation process of the invention.

### DETAILED DESCRIPTION

The ketonization of acetic acid with itself and other carboxylic acids, esters, or aldehydes is a valuable and high yield means to the synthesis of acetone and other methyl ketones. The ketonization of acetic acid to acetone co-produces one mole of water along with each mole of acetone produced. Moreover, although the yield of ketone from acetic acid can be over 99%, based on moles of acetic acid fed to the reactor to moles of ketone produced, small amounts of heavier organics, such as mesityl oxide, mesitylene, isophorone, methyl ethyl ketone, methyl propyl ketone, and isopropyl isopropenyl ketone, are formed as by-products of the ketonization reaction. A crude acetone stream derived from ketonization of acetic acid will comprise acetone, water, and by-product organics. There is now provided a process for purifying acetone comprising: (a) feeding a crude product mixture from an acetic acid ketonization reaction product stream comprising acetone, water, an impurity and ketonization reaction by-products to a distillation column; (b) withdrawing a liquid side draw stream from the distillation column; (c) separating the side draw stream into a water layer and an organic layer, and returning the water layer to the distillation column; and (d) recovering from the distillation column: (i) a lower boiling fraction comprising a purified acetone relative to the crude product mixture and (ii) a higher boiling fraction comprising a major amount of the water.

Surprisingly, it has been unexpectedly discovered that during the high temperature ketonization of acetic acid to acetone, the impurity comprising an acetic acid azeotrope or pinch-forming compounds in the feed acetic acid are not thermally decomposed to lighter species and do not promote fouling of the ketonization catalyst. These impurities after exposure to ketonization conditions retain similar boiling point characteristics as the original feed impurity compounds species and, as a consequence, do not interfere with the purification of the acetone product. Thus, acetyl byproduct feed streams contaminated with up to about 50 weight percent of impurities in which at least one of the impurities forms an azeotrope or pinch point with the acetic acid, can be converted to acetone by a ketonization process and the impurities subsequently separated by distillation from the acetone product. Such impurities comprise at least one azeotrope-forming compound selected from the group consisting of an alkyl aromatic hydrocarbon, a ketone, an aromatic ester, an acyclic ester, a terpene, a terpenoid, an acyclic unsaturated hydrocarbon, and combinations thereof.

As used herein the term “ketonization,” is understood to mean a process in which two carboxylic acids, carboxylic acid salts, or esters are converted to a ketone, carbon dioxide, and water, at an elevated temperature. The ketonization of carboxylic acids is well-known method for the production symmetrical and unsymmetrical ketones. Generally, ketonization is intended to be synonymous with the term “ketonic decarboxylation” and refers to a process in which ketone is formed from the decarboxylative condensation of two carboxylic acid molecules. The ketonization of acetic acid with itself and other carboxylic acids, esters, or aldehydes is a valuable and high yield means to the synthesis of acetone and other methyl ketones. The ketonization of acetic acid to acetone co-produces one mole of water along with each mole of acetone produced. Moreover, although the yield of ketone from acetic acid can be over 99%, small amounts of heavier organics, such as mesityl oxide, mesitylene, isophorone, methyl ethyl ketone, methyl propyl ketone, and 2,6-dimethylhepta-2,5-dien-4-one, are formed as by-products of the ketonization reaction. Thus, a crude acetone stream derived from ketonization of acetic acid will comprise acetone, water, and by-product organics such as, for example, methanol, methyl isobutyl ketone, mesityl oxide, mesitylene, isophorone, methyl ethyl ketone, methyl propyl ketone, diacetone alcohol, 2,6-dimethylhepta-2,5-dien-4-one, and combinations thereof.

The ketonization of acetic acid with itself and cross ketonization of acetic acid with higher carboxylic acids are well known routes for the production of acetone and higher methyl ketones. The general reactions for self- and cross-ketonization of acetic acid are:

### Self-Ketonization:

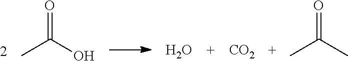

### Cross-Ketonization:

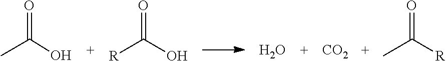

Unsymetric methyl ketones may be produced by co-feeding other carboxylic acid with acetic acid. For example, co-feeding propionic acid with acetic acid results in the formation of methyl ethyl ketone, n-butyric acid with acetic acid results in the formation of methyl propyl ketone, isobutyric acid with acetic acid results in formation of methyl isopropyl ketone.

The product ketones, exemplified by acetone, derived via ketonization of acetic acid in a manner described above, may undergo further reaction over the ketonization catalyst, following an aldol-like condensation/dehydration pathway to form higher α,β-unsaturated ketones, most notably mesityl oxide via condensation of acetone.

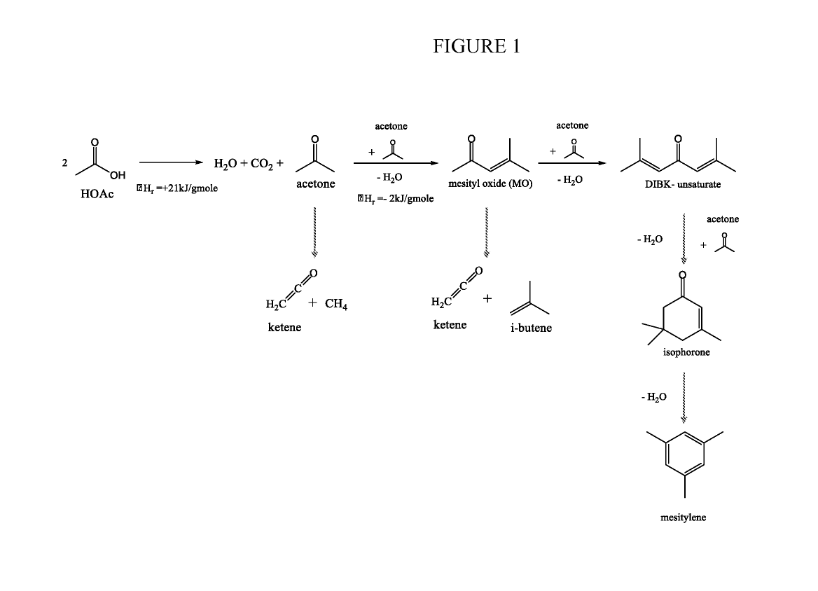

It has been found that mesityl oxide may undergo a further decomposition reaction to produce a reaction product comprising isobutylene. Although a relatively minor yield loss, typically comprising 0.02 to 0.8 mole percent conversion of the feed acetic acid to isobutylene, isobutylene is a highly volatile species, and is difficult to remove from the effluent carbon dioxide by-product stream in a cost effective manner. Moreover, the effluent by-product carbon dioxide stream may contain small amounts of unrecovered product ketone, such as, acetone, and other by-products resulting from side reactions, exemplified by methane, hydrogen, mesitylene, isophorone or by reactions of feed impurities with acetic acid, as exemplified by methyl ethyl ketone (via ketonization of acetic acid with traces of propionic acid). It is also undesirable to emit these compounds with the by-product carbon dioxide stream. A more complete reaction network for the ketonization of acetic acid to acetone is shown in FIG. 1. One skilled in the art will understand cross ketonization of acetic acid with higher carboxylic acids can produce acetone and higher molecular weight methyl ketones.

The invention also includes a process for purifying acetone comprising: a) vaporizing an acetyl feed stream comprising acetic acid, an impurity comprising at least one acetic acid azeotrope-forming compound, and 0-50 weight % water, optionally mixing steam with the vaporized acetyl feed stream to produce a vaporized feed mixture; b) superheating the vaporized feed mixture to produce a superheated feed mixture; c) contacting the superheated feed mixture with a metal oxide catalyst in a ketonization reactor to produce a crude product mixture comprising acetone, water, the impurity, carbon dioxide, and byproducts from the ketonization reaction; d) recovering condensable components of the crude product mixture to produce a recovered liquid crude acetone stream comprising acetone, water, an impurity and ketonization by-products and a gaseous off-gas stream; e) feeding the recovered liquid crude acetone stream to a distillation column; f) withdrawing a liquid side draw stream from the distillation column; g) separating the side draw stream into a water layer and an organic layer, and returning the water layer to the distillation column; and h) recovering from the distillation column: (i) a lower boiling fraction comprising a purified acetone relative to the crude product mixture and (ii) a higher boiling fraction comprising a major amount of the water.

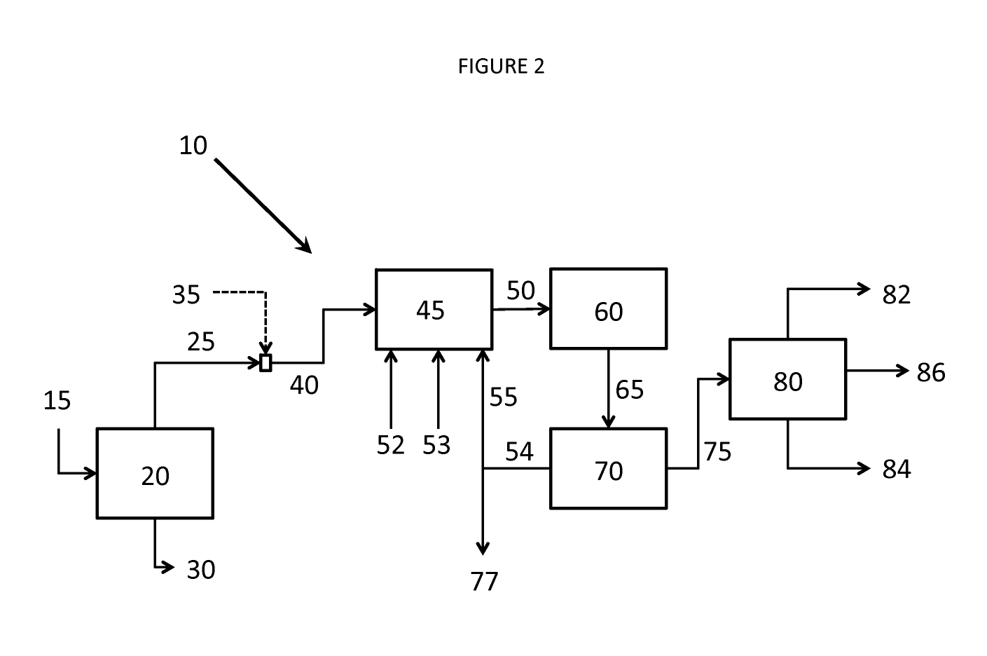

With reference to FIG. 2, the present invention is described in greater detail. The ketonization process 10 includes feeding an acetyl stream 15 comprising acetic acid, an impurity comprising at least one acetic acid azeotrope-forming compound and 0-50 weight % water, to a vaporization unit 20, wherein a fraction, typically 75 to 99%, of the acetyl feed stream 15 is vaporized by boiling against steam, to produce vaporized acetyl stream 25. A portion of the feed acid that is not vaporized is removed from the vaporization unit 20 as sludge via line 30. Vaporized acetyl stream 25 is optionally mixed with steam 35 for further dilution of the feed acid to produce vaporized wet acid feed mixture 40. Vaporized wet acid feed mixture 40 is further superheated to the desired reaction inlet temperature in a feed superheater furnace 45 to produce superheated feed mixture 50. Heat is provided to the furnace by combustion of fuel 52 with an oxygen-containing stream 53, which may be diluted for temperature control by at least a portion of by-product carbon dioxide stream 54 via conduit 55. The superheated acid feed mixture 50 is passed through ketonization reactor 60, wherein the acetic acid and other reactive feed molecules, if present, are converted over a heterogeneous ketonization catalyst to produce a crude product mixture 65 comprising acetone, water, carbon dioxide, unreacted acetic acid, the impurity having at least one acetic acid azeotrope-forming compound, and other minor by-products. Crude product mixture 65, is cooled and separated in recovery zone 70 to produce a liquid crude acetone stream 75, comprising the majority of the acetone, water, impurities and heavy by-products; and gaseous off-gas stream 54 comprising carbon dioxide, isobutylene, methane, hydrogen, other minor VOC's, and traces of acetone and higher by-products. Gaseous off-gas stream 54, may be sent in its entirety via conduit 55 to the superheater or furnace 45, or a portion emitted directly via conduit 77. Typically during, normal operation all of stream 54 will be sent to the superheater 45 for combustion of volatile organic compounds (VOCs), although at start up, or during furnace upsets, a fraction or all of stream 54 may exit the process via stream 77 without further treatment. The liquid crude acetone stream 75 is further purified in distillation zone 80 to produce a lower boiling fraction 82 comprising a purified acetone, relative to the crude product mixture and (ii) a higher boiling fraction 84 comprising a major amount of the water. As used herein, the term “purified acetone, relative to the crude product mixture” means that the concentration in weight %, based on the total weight of the respective stream, of acetone in the lower boiling fraction is higher than that in the crude product mixture and the concentration of impurities, including water, is lower in the lower boiling fraction than that in the crude product mixture.

Advantageously, the acetic acid comprising the acetyl feed stream 15 can be, but is not limited to, a byproduct from one or more of the processes discussed above, i.e., acetylation of a compound selected from an alcohol, a polyol, cellulose, an amine, carboxylic acid, and an aromatic compound by contacting the compound with acetic anhydride. For example, the acetic acid utilized can be a byproduct from one or more of the following processes: acetylation of wood; acetylation of alcohols with acetic anhydride to form esters; carbonylation of methanol and methyl acetate to form acetic acid and acetic anhydride; preparation of 2,2,4,4-tetramethyl-1,3-cyclobutanediol from isobutyric anhydride via acetic anhydride exchange; preparation of ketenes and diketene from acetic acid; polymerization reactions, such as condensation of phenyl acetate monomers to produce polyesters or polycarbonates; preparation of fine chemicals and pharmaceuticals; preparation of carboxylic acid anhydrides from their corresponding acids by exchange with acetic anhydride; and acylation reactions. Examples for fine chemical and pharmaceutical products include but are not limited, to industrial production of ibuprofen and liquid crystal polymers.

It will be recognized that acetic acid from other sources may also be equally suitable for use in the process of the present invention. Such sources include, but are not limited to, acetaldehyde oxidation, ethylene oxidation, oxidative fermentation, and anaerobic biomass fermentation, producer waters from oil and gas production, Fischer-Tropsch derived acetic acid, and carbonaceous reforming.

The byproduct acetyl feed stream 15, containing a mixture of acetic acid, an impurity comprising at least one acetic acid azeotrope or pinch-forming impurity, and optionally, acetic anhydride, can be mixed with water to hydrolyze any acetic anhydride that is present to produce a wet acetic acid feed stream. The acetyl feed stream typically contains from about 40 to about 99 weight percent acetic acid, up to about 50 weight percent impurities, up to about 30 weight % water, and optionally up to about 10 weight percent acetic anhydride, wherein the weight percentages are based on total constituents in the feed stream. Accordingly, the acetyl feed stream 15 can have from about 40 to about 99 weight % acetic acid, or from about 70 to about 99 weight % acetic acid, or from about 86 to about 99 weight % acetic acid, or about 87 to about 99 weight % acetic acid, or about 88 to about 99 weight % acetic acid, or about 89 to about 99 weight % acetic acid, or about 90 to about 99 weight % acetic acid, or about 91 to about 99 weight % acetic acid, or about 92 to about 99 weight % acetic acid, or about 93 to about 99 weight % acetic acid, or about 94 to about 99 weight % acetic acid, or about 95 to about 99 weight % acetic acid, or about 96 to about 99 weight % acetic acid, or about 97 to about 99 weight % acetic acid, or about 98 to about 99 weight % acetic acid; and up to about 50 weight % impurities, or from about 100 ppm to about 50 weight % impurities, or from about 200 ppm to about 50 weight % impurities, or from about 500 ppm to about 50 weight % impurities, or from about 1000 ppm to about 50 weight % impurities, or from about 5000 ppm to about 50 weight % impurities, or from about 1 weight % to about 50 weight % impurities, or from about 2 weight % to about 50 weight % impurities, or from about 3 weight % to about 50 weight % impurities, or from about 4 weight % to about 50 weight % impurities, or from about 5 weight % to about 50 weight % impurities, or from about 6 weight % to about 50 weight % impurities, or from about 7 weight % to about 50 weight % impurities, or from about 8 weight % to about 50 weight % impurities, or from about 9 weight % to about 50 weight % impurities, or from about 10 weight % to about 50 weight % impurities, or from about 11 weight % to about 50 weight % impurities, or from about 12 weight % to about 50 weight % impurities, or from about 13 weight % to about 50 weight % impurities, or from about 14 weight % to about 50 weight % impurities; or from about 15 weight % to about 50 weight % impurities, or from about 16 weight % to about 50 weight % impurities, or from about 17 weight % to about 50 weight % impurities, or from about 18 weight % to about 50 weight % impurities, or from about 19 weight % to about 50 weight % impurities, or from about 20 weight % to about 50 weight % impurities, or from about 21 weight % to about 50 weight % impurities, or from about 22 weight % to about 50 weight % impurities, or from about 23 weight % to about 50 weight % impurities, or from about 24 weight % to about 50 weight % impurities, or from about 25 weight % to about 50 weight % impurities; 26 weight % to about 50 weight % impurities, or from about 27 weight % to about 50 weight % impurities, or from about 28 weight % to about 50 weight % impurities, or from about 29 weight % to about 50 weight % impurities, or from about 30 weight % to about 50 weight % impurities, or from about 31 weight % to about 50 weight % impurities, or from about 32 weight % to about 50 weight % impurities, or from about 33 weight % to about 50 weight % impurities, or from about 34 weight % to about 50 weight % impurities, or from about 35 weight % to about 50 weight % impurities, or from about 36 weight % to about 50 weight % impurities, or from about 37 weight % to about 50 weight % impurities, or from about 38 weight % to about 50 weight % impurities; or from about 39 weight % to about 50 weight % impurities, or from about 40 weight % to about 50 weight % impurities, or from about 41 weight % to about 50 weight % impurities, or from about 42 weight % to about 43 weight % impurities, or from about 44 weight % to about 50 weight % impurities, or from about 45 weight % to about 50 weight % impurities, or from about 46 weight % to about 50 weight % impurities, or from about 47 weight % to about 50 weight % impurities, or from about 48 weight % to about 50 weight % impurities, or from about 49 weight % to about 50 weight % impurities.

Optionally, the byproduct acetyl feed stream 15, may have from 200 ppm to about 15 weight % impurities, or from about 500 ppm to about 15 weight % impurities, or from about 1000 ppm to about 15 weight % impurities, or from about 5000 ppm to about 15 weight percent, or from 1 to about 15 weight % impurities, or from 2 to about 15 weight % impurities, or from 3 to about 15 weight % impurities, or from 4 to about 15 weight % impurities, or from 5 to about 15 weight % impurities, or from 6 to about 15 weight % impurities, or from 7 to about 15 weight % impurities, or from 8 to about 15 weight % impurities, or from 9 to about 15 weight % impurities, or from 10 to about 15 weight % impurities, or from 11 to about 15 weight % impurities, or from 12 to about 15 weight % impurities, or from 13 to about 15 weight % impurities, or from 14 to about 15 weight % impurities, or from 200 ppm to about 14 weight % impurities, or from about 500 ppm to about 14 weight % impurities, or from about 1000 ppm to about 14 weight % impurities, or from about 5000 ppm to about 14 weight percent, or from 1 to about 14 weight % impurities, or from 2 to about 14 weight % impurities, or from 3 to about 14 weight % impurities, or from 4 to about 14 weight % impurities, or from 5 to about 14 weight % impurities, or from 6 to about 14 weight % impurities, or from 7 to about 14 weight % impurities, or from 8 to about 14 weight % impurities, or from 9 to about 14 weight % impurities, or from 10 to about 14 weight % impurities, or from 11 to about 14 weight % impurities, or from 12 to about 14 weight % impurities, or from 13 to about 14 weight % impurities, or from 200 ppm to about 13 weight % impurities, or from about 500 ppm to about 15 weight % impurities, or from about 1000 ppm to about 13 weight % impurities, or from about 5000 ppm to about 13 weight percent, or from 1 to about 13 weight % impurities, or from 2 to about 13 weight % impurities, or from 3 to about 13 weight % impurities, or from 4 to about 13 weight % impurities, or from 5 to about 13 weight % impurities, or from 6 to about 13 weight % impurities, or from 7 to about 13 weight % impurities, or from 8 to about 13 weight % impurities, or from 9 to about 13 weight % impurities, or from 10 to about 13 weight % impurities, or from 11 to about 13 weight % impurities, or from 12 to about 13 weight % impurities, or from 200 ppm to about 12 weight % impurities, or from about 500 ppm to about 12 weight % impurities, or from about 1000 ppm to about 12 weight % impurities, or from about 5000 ppm to about 12 weight percent, or from 1 to about 12 weight % impurities, or from 2 to about 12 weight % impurities, or from 3 to about 12 weight % impurities, or from 4 to about 12 weight % impurities, or from 5 to about 12 weight % impurities, or from 6 to about 12 weight % impurities, or from 7 to about 12 weight % impurities, or from 8 to about 12 weight % impurities, or from 9 to about 12 weight % impurities, or from 10 to about 12 weight % impurities, or from 11 to about 12 weight % impurities, or from 200 ppm to about 11 weight % impurities, or from about 500 ppm to about 11 weight % impurities, or from about 1000 ppm to about 11 weight % impurities, or from about 5000 ppm to about 11 weight percent, or from 1 to about 11 weight % impurities, or from 2 to about 11 weight % impurities, or from 3 to about 11 weight % impurities, or from 4 to about 11 weight % impurities, or from 5 to about 11 weight % impurities, or from 6 to about 11 weight % impurities, or from 7 to about 11 weight % impurities, or from 8 to about 11 weight % impurities, or from 9 to about 11 weight % impurities, or from 10 to about 11 weight % impurities, or from 200 ppm to about 10 weight % impurities, or from about 500 ppm to about 10 weight % impurities, or from about 1000 ppm to about 10 weight % impurities, or from about 5000 ppm to about 10 weight percent, or from 1 to about 10 weight % impurities, or from 2 to about 10 weight % impurities, or from 3 to about 10 weight % impurities, or from 4 to about 10 weight % impurities, or from 5 to about 10 weight % impurities, or from 6 to about 10 weight % impurities, or from 7 to about 10 weight % impurities, or from 8 to about 10 weight % impurities, or from 9 to about 10 weight % impurities, or from 200 ppm to about 9 weight % impurities, or from about 500 ppm to about 9 weight % impurities, or from about 1000 ppm to about 9 weight % impurities, or from about 5000 ppm to about 9 weight percent, or from 1 to about 9 weight % impurities, or from 2 to about 9 weight % impurities, or from 3 to about 9 weight % impurities, or from 4 to about 9 weight % impurities, or from 5 to about 9 weight % impurities, or from 6 to about 9 weight % impurities, or from 7 to about 9 weight % impurities, or from 8 to about 9 weight % impurities, or from 200 ppm to about 8 weight % impurities, or from about 500 ppm to about 8 weight % impurities, or from about 1000 ppm to about 8 weight % impurities, or from about 5000 ppm to about 8 weight percent, or from 1 to about 8 weight % impurities, or from 2 to about 8 weight % impurities, or from 3 to about 8 weight % impurities, or from 4 to about 8 weight % impurities, or from 5 to about 8 weight % impurities, or from 6 to about 8 weight % impurities, or from 7 to about 8 weight % impurities, or from 200 ppm to about 7 weight % impurities, or from about 500 ppm to about 7 weight % impurities, or from about 1000 ppm to about 7 weight % impurities, or from about 5000 ppm to about 7 weight percent, or from 1 to about 7 weight % impurities, or from 2 to about 7 weight % impurities, or from 3 to about 7 weight % impurities, or from 4 to about 7 weight % impurities, or from 5 to about 7 weight % impurities, or from 6 to about 7 weight % impurities, or from 200 ppm to about 6 weight % impurities, or from about 500 ppm to about 6 weight % impurities, or from about 1000 ppm to about 6 weight % impurities, or from about 5000 ppm to about 6 weight percent, or from 1 to about 6 weight % impurities, or from 2 to about 6 weight % impurities, or from 3 to about 6 weight % impurities, or from 4 to about 6 weight % impurities, or from 200 ppm to about 5 weight % impurities, or from about 500 ppm to about 5 weight % impurities, or from about 1000 ppm to about 5 weight % impurities, or from about 5000 ppm to about 5 weight percent, or from 1 to about 5 weight % impurities, or from 2 to about 5 weight % impurities, or from 3 to about 5 weight % impurities, or from 4 to about 5 weight % impurities, or from 200 ppm to about 4 weight % impurities, or from about 500 ppm to about 4 weight % impurities, or from about 1000 ppm to about 4 weight % impurities, or from about 5000 ppm to about 4 weight percent, or from 1 to about 4 weight % impurities, or from 2 to about 4 weight % impurities, or from 3 to about 4 weight % impurities, or from 200 ppm to about 3 weight % impurities, or from about 500 ppm to about 3 weight % impurities, or from about 1000 ppm to about 3 weight % impurities, or from about 5000 ppm to about 3 weight percent, or from 1 to about 3 weight % impurities, or from 2 to about 3 weight % impurities, or from 200 ppm to about 2 weight % impurities, or from about 500 ppm to about 2 weight % impurities, or from about 1000 ppm to about 2 weight % impurities, or from about 5000 ppm to about 2 weight percent, or from 1 to about 2 weight % impurities, wherein the weight percentages presented above are based on the total weight of the constituents of the feed stream 15.

Due to excessive fouling of the vaporizer 20, superheater 45, and reactor 60, it is not desirable to feed anhydrous acetic acid or acetic anhydride. Rather, the acetyl feed stream 15 is mixed with sufficient water to hydrolyze any acetic anhydride that may be present prior to introducing the acetyl feed stream 15 to the vaporizer 20. Accordingly, the feed stream 15 can be mixed with water to bring the final concentration of water in the acetyl feed stream 15 up to about 80 weight % water, or from about 10 weight % to about 80 weight % water, or from about 15 weight % to about 80 weight % water, or from about 20 weight % to about 80 weight % water, or from about 25 weight % to about 80 weight % water, or from about 30 weight % to about 80 weight % water, or from about 35 weight % to about 80 weight % water, or from about 40 weight % to about 80 weight % water, or from about 45 weight % to about 80 weight % water, or from about 50 weight % to about 80 weight % water, or from about 55 weight % to about 80 weight % water, or from about 60 weight % to about 80 weight % water, or from about 65 weight % to about 80 weight % water, or from about 70 weight % to about 80 weight % water, or from about 75 weight % to about 80 weight % water, wherein the weight percentage is based on the total weight of the feed stream 15.

The feed stream 15 may optionally further include up to about 75 weight % water, or up to about 70 weight % water, or up to about 65 weight % water, or up to about 60 weight % water, or up to about 55 weight % water, or up to about 50 weight % water, or up to about 45 weight % water, or up to about 40 weight % water, or up to about 35 weight % water, or up to about 30 weight % water, or up to about 25 weight % water, or up to about 20 weight % water, or up to about 15 weight % water, or up to about 10 weight % water, wherein the weight percentage is based on the total weight of the constituents of the feed stream 15. It should be generally understood that in the ranges specified above the term “up to” includes from 0 to the delineated end point, and includes all ranges in between. Such ranges include 0 to 80, 1 to 80, 2 to 80, 3 to 80, 4 to 80, 5 to 80, 6 to 80, 7 to 80, 8 to 80, 9 to 80, 10 to 80, 11 to 80, 12 to 80, 13 to 80, 14 to 80, 15 to 80, 16 to 80, 17 to 80, 18 to 80, 19 to 80, 20 to 80, 21 to 80, 22 to 80, 23 to 80, 24 to 80, 25 to 80, 26 to 80, 27 to 80, 28 to 80, 29 to 80, 30 to 80, 31 to 80, 32 to 80, 33 to 80, 34 to 80, 35 to 80, 36 to 80, 37 to 80, 38 to 80, 39 to 80, 40 to 80, 41 to 80, 42 to 80, 43 to 80, 44 to 80, 45 to 80, 46 to 80, 47 to 80, 48 to 80, 49 to 80, 50 to 80, 51 to 80, 52 to 80, 53 to 80, 54 to 80, 55 to 80, 56 to 80, 57 to 80, 58 to 80, 59 to 80, 60 to 80, 61 to 80, 62 to 80, 63 to 80, 64 to 80, 65 to 80, 66 to 80, 67 to 80, 68 to 80, 69 to 80, 70 to 80, 71 to 80, 72 to 80, 73 to 80, 74 to 80, 75 to 80, 76 to 80, 77 to 80, 78 to 80, 79 to 80, 0 to 79, 1 to 79, 2 to 79, 3 to 79, 4 to 79, 5 to 79, 6 to 79, 7 to 79, 8 to 79, 9 to 79, 10 to 79, 11 to 79, 12 to 79, 13 to 79, 14 to 79, 15 to 79, 16 to 79, 17 to 79, 18 to 79, 19 to 79, 20 to 79, 21 to 79, 22 to 79, 23 to 79, 24 to 79, 25 to 79, 26 to 79, 27 to 79, 28 to 79, 29 to 79, 30 to 79, 31 to 79, 32 to 79, 33 to 79, 34 to 79, 35 to 79, 36 to 79, 37 to 79, 38 to 79, 39 to 79, 40 to 79, 41 to 79, 42 to 79, 43 to 79, 44 to 79, 45 to 79, 46 to 79, 47 to 79, 48 to 79, 49 to 79, 50 to 79, 51 to 79, 52 to 79, 53 to 79, 54 to 79, 55 to 79, 56 to 79, 57 to 79, 58 to 79, 59 to 79, 60 to 79, 61 to 79, 62 to 79, 63 to 79, 64 to 79, 65 to 79, 66 to 79, 67 to 79, 68 to 79, 69 to 79, 70 to 79, 71 to 79, 72 to 79, 73 to 79, 74 to 79, 75 to 79, 76 to 79, 77 to 79, 78 to 79, 0 to 78, 1 to 78, 2 to 78, 3 to 78, 4 to 78, 5 to 78, 6 to 78, 7 to 78, 8 to 78, 9 to 78, 10 to 78, 11 to 78, 12 to 78, 13 to 78, 14 to 78, 15 to 78, 16 to 78, 17 to 78, 18 to 78, 19 to 78, 20 to 78, 21 to 78, 22 to 78, 23 to 78, 24 to 78, 25 to 78, 26 to 78, 27 to 78, 28 to 78, 29 to 78, 30 to 78, 31 to 78, 32 to 78, 33 to 78, 34 to 78, 35 to 78, 36 to 78, 37 to 78, 38 to 78, 39 to 78, 40 to 78, 41 to 78, 42 to 78, 43 to 78, 44 to 78, 45 to 78, 46 to 78, 47 to 78, 48 to 78, 49 to 78, 50 to 78, 51 to 78, 52 to 78, 53 to 78, 54 to 78, 55 to 78, 56 to 78, 57 to 78, 58 to 78, 59 to 78, 60 to 78, 61 to 78, 62 to 78, 63 to 78, 64 to 78, 65 to 78, 66 to 78, 67 to 78, 68 to 78, 69 to 78, 70 to 78, 71 to 78, 72 to 78, 73 to 78, 74 to 78, 75 to 78, 76 to 78, 77 to 78, 0 to 77, 1 to 77, 2 to 77, 3 to 77, 4 to 77, 5 to 77, 6 to 77, 7 to 77, 8 to 77, 9 to 77, 10 to 77, 11 to 77, 12 to 77, 13 to 77, 14 to 77, 15 to 77, 16 to 77, 17 to 77, 18 to 77, 19 to 77, 20 to 77, 21 to 77, 22 to 77, 23 to 77, 24 to 77, 25 to 77, 26 to 77, 27 to 77, 28 to 77, 29 to 77, 30 to 77, 31 to 77, 32 to 77, 33 to 77, 34 to 77, 35 to 77, 36 to 77, 37 to 77, 38 to 77, 39 to 77, 40 to 77, 41 to 77, 42 to 77, 43 to 77, 44 to 77, 45 to 77, 46 to 77, 47 to 77, 48 to 77, 49 to 77, 50 to 77, 51 to 77, 52 to 77, 53 to 77, 54 to 77, 55 to 77, 56 to 77, 57 to 77, 58 to 77, 59 to 77, 60 to 77, 61 to 77, 62 to 77, 63 to 77, 64 to 77, 65 to 77, 66 to 77, 67 to 77, 68 to 77, 69 to 77, 70 to 77, 71 to 77, 72 to 77, 73 to 77, 74 to 77, 75 to 77, 76 to 77, 0 to 76, 1 to 76, 2 to 76, 3 to 76, 4 to 76, 5 to 76, 6 to 76, 7 to 76, 8 to 76, 9 to 76, 10 to 76, 11 to 76, 12 to 76, 13 to 76, 14 to 76, 15 to 76, 16 to 76, 17 to 76, 18 to 76, 19 to 76, 20 to 76, 21 to 76, 22 to 76, 23 to 76, 24 to 76, 25 to 76, 26 to 76, 27 to 76, 28 to 76, 29 to 76, 30 to 76, 31 to 76, 32 to 76, 33 to 76, 34 to 76, 35 to 76, 36 to 76, 37 to 76, 38 to 76, 39 to 76, 40 to 76, 41 to 76, 42 to 76, 43 to 76, 44 to 76, 45 to 76, 46 to 76, 47 to 76, 48 to 76, 49 to 76, 50 to 76, 51 to 76, 52 to 76, 53 to 76, 54 to 76, 55 to 76, 56 to 76, 57 to 76, 58 to 76, 59 to 76, 60 to 76, 61 to 76, 62 to 76, 63 to 76, 64 to 76, 65 to 76, 66 to 76, 67 to 76, 68 to 76, 69 to 76, 70 to 76, 71 to 76, 72 to 76, 73 to 76, 74 to 76, 75 to 76, 0 to 75, 1 to 75, 2 to 75, 3 to 75, 4 to 75, 5 to 75, 6 to 75, 7 to 75, 8 to 75, 9 to 75, 10 to 75, 11 to 75, 12 to 75, 13 to 75, 14 to 75, 15 to 75, 16 to 75, 17 to 75, 18 to 75, 19 to 75, 20 to 75, 21 to 75, 22 to 75, 23 to 75, 24 to 75, 25 to 75, 26 to 75, 27 to 75, 28 to 75, 29 to 75, 30 to 75, 31 to 75, 32 to 75, 33 to 75, 34 to 75, 35 to 75, 36 to 75, 37 to 75, 38 to 75, 39 to 75, 40 to 75, 41 to 75, 42 to 75, 43 to 75, 44 to 75, 45 to 75, 46 to 75, 47 to 75, 48 to 75, 49 to 75, 50 to 75, 51 to 75, 52 to 75, 53 to 75, 54 to 75, 55 to 75, 56 to 75, 57 to 75, 58 to 75, 59 to 75, 60 to 75, 61 to 75, 62 to 75, 63 to 75, 64 to 75, 65 to 75, 66 to 75, 67 to 75, 68 to 75, 69 to 75, 70 to 75, 71 to 75, 72 to 75, 73 to 75, 74 to 75, 0 to 74, 1 to 74, 2 to 74, 3 to 74, 4 to 74, 5 to 74, 6 to 74, 7 to 74, 8 to 74, 9 to 74, 10 to 74, 11 to 74, 12 to 74, 13 to 74, 14 to 74, 15 to 74, 16 to 74, 17 to 74, 18 to 74, 19 to 74, 20 to 74, 21 to 74, 22 to 74, 23 to 74, 24 to 74, 25 to 74, 26 to 74, 27 to 74, 28 to 74, 29 to 74, 30 to 74, 31 to 74, 32 to 74, 33 to 74, 34 to 74, 35 to 74, 36 to 74, 37 to 74, 38 to 74, 39 to 74, 40 to 74, 41 to 74, 42 to 74, 43 to 74, 44 to 74, 45 to 74, 46 to 74, 47 to 74, 48 to 74, 49 to 74, 50 to 74, 51 to 74, 52 to 74, 53 to 74, 54 to 74, 55 to 74, 56 to 74, 57 to 74, 58 to 74, 59 to 74, 60 to 74, 61 to 74, 62 to 74, 63 to 74, 64 to 74, 65 to 74, 66 to 74, 67 to 74, 68 to 74, 69 to 74, 70 to 74, 71 to 74, 72 to 74, 73 to 74, 0 to 73, 1 to 73, 2 to 73, 3 to 73, 4 to 73, 5 to 73, 6 to 73, 7 to 73, 8 to 73, 9 to 73, 10 to 73, 11 to 73, 12 to 73, 13 to 73, 14 to 73, 15 to 73, 16 to 73, 17 to 73, 18 to 73, 19 to 73, 20 to 73, 21 to 73, 22 to 73, 23 to 73, 24 to 73, 25 to 73, 26 to 73, 27 to 73, 28 to 73, 29 to 73, 30 to 73, 31 to 73, 32 to 73, 33 to 73, 34 to 73, 35 to 73, 36 to 73, 37 to 73, 38 to 73, 39 to 73, 40 to 73, 41 to 73, 42 to 73, 43 to 73, 44 to 73, 45 to 73, 46 to 73, 47 to 73, 48 to 73, 49 to 73, 50 to 73, 51 to 73, 52 to 73, 53 to 73, 54 to 73, 55 to 73, 56 to 73, 57 to 73, 58 to 73, 59 to 73, 60 to 73, 61 to 73, 62 to 73, 63 to 73, 64 to 73, 65 to 73, 66 to 73, 67 to 73, 68 to 73, 69 to 73, 70 to 73, 71 to 73, 72 to 73, 0 to 72, 1 to 72, 2 to 72, 3 to 72, 4 to 72, 5 to 72, 6 to 72, 7 to 72, 8 to 72, 9 to 72, 10 to 72, 11 to 72, 12 to 72, 13 to 72, 14 to 72, 15 to 72, 16 to 72, 17 to 72, 18 to 72, 19 to 72, 20 to 72, 21 to 72, 22 to 72, 23 to 72, 24 to 72, 25 to 72, 26 to 72, 27 to 72, 28 to 72, 29 to 72, 30 to 72, 31 to 72, 32 to 72, 33 to 72, 34 to 72, 35 to 72, 36 to 72, 37 to 72, 38 to 72, 39 to 72, 40 to 72, 41 to 72, 42 to 72, 43 to 72, 44 to 72, 45 to 72, 46 to 72, 47 to 72, 48 to 72, 49 to 72, 50 to 72, 51 to 72, 52 to 72, 53 to 72, 54 to 72, 55 to 72, 56 to 72, 57 to 72, 58 to 72, 59 to 72, 60 to 72, 61 to 72, 62 to 72, 63 to 72, 64 to 72, 65 to 72, 66 to 72, 67 to 72, 68 to 72, 69 to 72, 70 to 72, 71 to 72, 0 to 71, 1 to 71, 2 to 71, 3 to 71, 4 to 71, 5 to 71, 6 to 71, 7 to 71, 8 to 71, 9 to 71, 10 to 71, 11 to 71, 12 to 71, 13 to 71, 14 to 71, 15 to 71, 16 to 71, 17 to 71, 18 to 71, 19 to 71, 20 to 71, 21 to 71, 22 to 71, 23 to 71, 24 to 71, 25 to 71, 26 to 71, 27 to 71, 28 to 71, 29 to 71, 30 to 71, 31 to 71, 32 to 71, 33 to 71, 34 to 71, 35 to 71, 36 to 71, 37 to 71, 38 to 71, 39 to 71, 40 to 71, 41 to 71, 42 to 71, 43 to 71, 44 to 71, 45 to 71, 46 to 71, 47 to 71, 48 to 71, 49 to 71, 50 to 71, 51 to 71, 52 to 71, 53 to 71, 54 to 71, 55 to 71, 56 to 71, 57 to 71, 58 to 71, 59 to 71, 60 to 71, 61 to 71, 62 to 71, 63 to 71, 64 to 71, 65 to 71, 66 to 71, 67 to 71, 68 to 71, 69 to 71, 70 to 71, 0 to 70, 1 to 70, 2 to 70, 3 to 70, 4 to 70, 5 to 70, 6 to 70, 7 to 70, 8 to 70, 9 to 70, 10 to 70, 11 to 70, 12 to 70, 13 to 70, 14 to 70, 15 to 70, 16 to 70, 17 to 70, 18 to 70, 19 to 70, 20 to 70, 21 to 70, 22 to 70, 23 to 70, 24 to 70, 25 to 70, 26 to 70, 27 to 70, 28 to 70, 29 to 70, 30 to 70, 31 to 70, 32 to 70, 33 to 70, 34 to 70, 35 to 70, 36 to 70, 37 to 70, 38 to 70, 39 to 70, 40 to 70, 41 to 70, 42 to 70, 43 to 70, 44 to 70, 45 to 70, 46 to 70, 47 to 70, 48 to 70, 49 to 70, 50 to 70, 51 to 70, 52 to 70, 53 to 70, 54 to 70, 55 to 70, 56 to 70, 57 to 70, 58 to 70, 59 to 70, 60 to 70, 61 to 70, 62 to 70, 63 to 70, 64 to 70, 65 to 70, 66 to 70, 67 to 70, 68 to 70, 69 to 70, 0 to 69, 1 to 69, 2 to 69, 3 to 69, 4 to 69, 5 to 69, 6 to 69, 7 to 69, 8 to 69, 9 to 69, 10 to 69, 11 to 69, 12 to 69, 13 to 69, 14 to 69, 15 to 69, 16 to 69, 17 to 69, 18 to 69, 19 to 69, 20 to 69, 21 to 69, 22 to 69, 23 to 69, 24 to 69, 25 to 69, 26 to 69, 27 to 69, 28 to 69, 29 to 69, 30 to 69, 31 to 69, 32 to 69, 33 to 69, 34 to 69, 35 to 69, 36 to 69, 37 to 69, 38 to 69, 39 to 69, 40 to 69, 41 to 69, 42 to 69, 43 to 69, 44 to 69, 45 to 69, 46 to 69, 47 to 69, 48 to 69, 49 to 69, 50 to 69, 51 to 69, 52 to 69, 53 to 69, 54 to 69, 55 to 69, 56 to 69, 57 to 69, 58 to 69, 59 to 69, 60 to 69, 61 to 69, 62 to 69, 63 to 69, 64 to 69, 65 to 69, 66 to 69, 67 to 69, 68 to 69, 0 to 68, 1 to 68, 2 to 68, 3 to 68, 4 to 68, 5 to 68, 6 to 68, 7 to 68, 8 to 68, 9 to 68, 10 to 68, 11 to 68, 12 to 68, 13 to 68, 14 to 68, 15 to 68, 16 to 68, 17 to 68, 18 to 68, 19 to 68, 20 to 68, 21 to 68, 22 to 68, 23 to 68, 24 to 68, 25 to 68, 26 to 68, 27 to 68, 28 to 68, 29 to 68, 30 to 68, 31 to 68, 32 to 68, 33 to 68, 34 to 68, 35 to 68, 36 to 68, 37 to 68, 38 to 68, 39 to 68, 40 to 68, 41 to 68, 42 to 68, 43 to 68, 44 to 68, 45 to 68, 46 to 68, 47 to 68, 48 to 68, 49 to 68, 50 to 68, 51 to 68, 52 to 68, 53 to 68, 54 to 68, 55 to 68, 56 to 68, 57 to 68, 58 to 68, 59 to 68, 60 to 68, 61 to 68, 62 to 68, 63 to 68, 64 to 68, 65 to 68, 66 to 68, 67 to 68, 0 to 67, 1 to 67, 2 to 67, 3 to 67, 4 to 67, 5 to 67, 6 to 67, 7 to 67, 8 to 67, 9 to 67, 10 to 67, 11 to 67, 12 to 67, 13 to 67, 14 to 67, 15 to 67, 16 to 67, 17 to 67, 18 to 67, 19 to 67, 20 to 67, 21 to 67, 22 to 67, 23 to 67, 24 to 67, 25 to 67, 26 to 67, 27 to 67, 28 to 67, 29 to 67, 30 to 67, 31 to 67, 32 to 67, 33 to 67, 34 to 67, 35 to 67, 36 to 67, 37 to 67, 38 to 67, 39 to 67, 40 to 67, 41 to 67, 42 to 67, 43 to 67, 44 to 67, 45 to 67, 46 to 67, 47 to 67, 48 to 67, 49 to 67, 50 to 67, 51 to 67, 52 to 67, 53 to 67, 54 to 67, 55 to 67, 56 to 67, 57 to 67, 58 to 67, 59 to 67, 60 to 67, 61 to 67, 62 to 67, 63 to 67, 64 to 67, 65 to 67, 66 to 67, 0 to 66, 1 to 66, 2 to 66, 3 to 66, 4 to 66, 5 to 66, 6 to 66, 7 to 66, 8 to 66, 9 to 66, 10 to 66, 11 to 66, 12 to 66, 13 to 66, 14 to 66, 15 to 66, 16 to 66, 17 to 66, 18 to 66, 19 to 66, 20 to 66, 21 to 66, 22 to 66, 23 to 66, 24 to 66, 25 to 66, 26 to 66, 27 to 66, 28 to 66, 29 to 66, 30 to 66, 31 to 66, 32 to 66, 33 to 66, 34 to 66, 35 to 66, 36 to 66, 37 to 66, 38 to 66, 39 to 66, 40 to 66, 41 to 66, 42 to 66, 43 to 66, 44 to 66, 45 to 66, 46 to 66, 47 to 66, 48 to 66, 49 to 66, 50 to 66, 51 to 66, 52 to 66, 53 to 66, 54 to 66, 55 to 66, 56 to 66, 57 to 66, 58 to 66, 59 to 66, 60 to 66, 61 to 66, 62 to 66, 63 to 66, 64 to 66, 65 to 66, 0 to 65, 1 to 65, 2 to 65, 3 to 65, 4 to 65, 5 to 65, 6 to 65, 7 to 65, 8 to 65, 9 to 65, 10 to 65, 11 to 65, 12 to 65, 13 to 65, 14 to 65, 15 to 65, 16 to 65, 17 to 65, 18 to 65, 19 to 65, 20 to 65, 21 to 65, 22 to 65, 23 to 65, 24 to 65, 25 to 65, 26 to 65, 27 to 65, 28 to 65, 29 to 65, 30 to 65, 31 to 65, 32 to 65, 33 to 65, 34 to 65, 35 to 65, 36 to 65, 37 to 65, 38 to 65, 39 to 65, 40 to 65, 41 to 65, 42 to 65, 43 to 65, 44 to 65, 45 to 65, 46 to 65, 47 to 65, 48 to 65, 49 to 65, 50 to 65, 51 to 65, 52 to 65, 53 to 65, 54 to 65, 55 to 65, 56 to 65, 57 to 65, 58 to 65, 59 to 65, 60 to 65, 61 to 65, 62 to 65, 63 to 65, 64 to 65, 0 to 64, 1 to 64, 2 to 64, 3 to 64, 4 to 64, 5 to 64, 6 to 64, 7 to 64, 8 to 64, 9 to 64, 10 to 64, 11 to 64, 12 to 64, 13 to 64, 14 to 64, 15 to 64, 16 to 64, 17 to 64, 18 to 64, 19 to 64, 20 to 64, 21 to 64, 22 to 64, 23 to 64, 24 to 64, 25 to 64, 26 to 64, 27 to 64, 28 to 64, 29 to 64, 30 to 64, 31 to 64, 32 to 64, 33 to 64, 34 to 64, 35 to 64, 36 to 64, 37 to 64, 38 to 64, 39 to 64, 40 to 64, 41 to 64, 42 to 64, 43 to 64, 44 to 64, 45 to 64, 46 to 64, 47 to 64, 48 to 64, 49 to 64, 50 to 64, 51 to 64, 52 to 64, 53 to 64, 54 to 64, 55 to 64, 56 to 64, 57 to 64, 58 to 64, 59 to 64, 60 to 64, 61 to 64, 62 to 64, 63 to 64, 0 to 63, 1 to 63, 2 to 63, 3 to 63, 4 to 63, 5 to 63, 6 to 63, 7 to 63, 8 to 63, 9 to 63, 10 to 63, 11 to 63, 12 to 63, 13 to 63, 14 to 63, 15 to 63, 16 to 63, 17 to 63, 18 to 63, 19 to 63, 20 to 63, 21 to 63, 22 to 63, 23 to 63, 24 to 63, 25 to 63, 26 to 63, 27 to 63, 28 to 63, 29 to 63, 30 to 63, 31 to 63, 32 to 63, 33 to 63, 34 to 63, 35 to 63, 36 to 63, 37 to 63, 38 to 63, 39 to 63, 40 to 63, 41 to 63, 42 to 63, 43 to 63, 44 to 63, 45 to 63, 46 to 63, 47 to 63, 48 to 63, 49 to 63, 50 to 63, 51 to 63, 52 to 63, 53 to 63, 54 to 63, 55 to 63, 56 to 63, 57 to 63, 58 to 63, 59 to 63, 60 to 63, 61 to 63, 62 to 63, 0 to 62, 1 to 62, 2 to 62, 3 to 62, 4 to 62, 5 to 62, 6 to 62, 7 to 62, 8 to 62, 9 to 62, 10 to 62, 11 to 62, 12 to 62, 13 to 62, 14 to 62, 15 to 62, 16 to 62, 17 to 62, 18 to 62, 19 to 62, 20 to 62, 21 to 62, 22 to 62, 23 to 62, 24 to 62, 25 to 62, 26 to 62, 27 to 62, 28 to 62, 29 to 62, 30 to 62, 31 to 62, 32 to 62, 33 to 62, 34 to 62, 35 to 62, 36 to 62, 37 to 62, 38 to 62, 39 to 62, 40 to 62, 41 to 62, 42 to 62, 43 to 62, 44 to 62, 45 to 62, 46 to 62, 47 to 62, 48 to 62, 49 to 62, 50 to 62, 51 to 62, 52 to 62, 53 to 62, 54 to 62, 55 to 62, 56 to 62, 57 to 62, 58 to 62, 59 to 62, 60 to 62, 61 to 62, 0 to 61, 1 to 61, 2 to 61, 3 to 61, 4 to 61, 5 to 61, 6 to 61, 7 to 61, 8 to 61, 9 to 61, 10 to 61, 11 to 61, 12 to 61, 13 to 61, 14 to 61, 15 to 61, 16 to 61, 17 to 61, 18 to 61, 19 to 61, 20 to 61, 21 to 61, 22 to 61, 23 to 61, 24 to 61, 25 to 61, 26 to 61, 27 to 61, 28 to 61, 29 to 61, 30 to 61, 31 to 61, 32 to 61, 33 to 61, 34 to 61, 35 to 61, 36 to 61, 37 to 61, 38 to 61, 39 to 61, 40 to 61, 41 to 61, 42 to 61, 43 to 61, 44 to 61, 45 to 61, 46 to 61, 47 to 61, 48 to 61, 49 to 61, 50 to 61, 51 to 61, 52 to 61, 53 to 61, 54 to 61, 55 to 61, 56 to 61, 57 to 61, 58 to 61, 59 to 61, 60 to 61, 0 to 60, 1 to 60, 2 to 60, 3 to 60, 4 to 60, 5 to 60, 6 to 60, 7 to 60, 8 to 60, 9 to 60, 10 to 60, 11 to 60, 12 to 60, 13 to 60, 14 to 60, 15 to 60, 16 to 60, 17 to 60, 18 to 60, 19 to 60, 20 to 60, 21 to 60, 22 to 60, 23 to 60, 24 to 60, 25 to 60, 26 to 60, 27 to 60, 28 to 60, 29 to 60, 30 to 60, 31 to 60, 32 to 60, 33 to 60, 34 to 60, 35 to 60, 36 to 60, 37 to 60, 38 to 60, 39 to 60, 40 to 60, 41 to 60, 42 to 60, 43 to 60, 44 to 60, 45 to 60, 46 to 60, 47 to 60, 48 to 60, 49 to 60, 50 to 60, 51 to 60, 52 to 60, 53 to 60, 54 to 60, 55 to 60, 56 to 60, 57 to 60, 58 to 60, 59 to 60, 0 to 59, 1 to 59, 2 to 59, 3 to 59, 4 to 59, 5 to 59, 6 to 59, 7 to 59, 8 to 59, 9 to 59, 10 to 59, 11 to 59, 12 to 59, 13 to 59, 14 to 59, 15 to 59, 16 to 59, 17 to 59, 18 to 59, 19 to 59, 20 to 59, 21 to 59, 22 to 59, 23 to 59, 24 to 59, 25 to 59, 26 to 59, 27 to 59, 28 to 59, 29 to 59, 30 to 59, 31 to 59, 32 to 59, 33 to 59, 34 to 59, 35 to 59, 36 to 59, 37 to 59, 38 to 59, 39 to 59, 40 to 59, 41 to 59, 42 to 59, 43 to 59, 44 to 59, 45 to 59, 46 to 59, 47 to 59, 48 to 59, 49 to 59, 50 to 59, 51 to 59, 52 to 59, 53 to 59, 54 to 59, 55 to 59, 56 to 59, 57 to 59, 58 to 59, 0 to 58, 1 to 58, 2 to 58, 3 to 58, 4 to 58, 5 to 58, 6 to 58, 7 to 58, 8 to 58, 9 to 58, 10 to 58, 11 to 58, 12 to 58, 13 to 58, 14 to 58, 15 to 58, 16 to 58, 17 to 58, 18 to 58, 19 to 58, 20 to 58, 21 to 58, 22 to 58, 23 to 58, 24 to 58, 25 to 58, 26 to 58, 27 to 58, 28 to 58, 29 to 58, 30 to 58, 31 to 58, 32 to 58, 33 to 58, 34 to 58, 35 to 58, 36 to 58, 37 to 58, 38 to 58, 39 to 58, 40 to 58, 41 to 58, 42 to 58, 43 to 58, 44 to 58, 45 to 58, 46 to 58, 47 to 58, 48 to 58, 49 to 58, 50 to 58, 51 to 58, 52 to 58, 53 to 58, 54 to 58, 55 to 58, 56 to 58, 57 to 58, 0 to 57, 1 to 57, 2 to 57, 3 to 57, 4 to 57, 5 to 57, 6 to 57, 7 to 57, 8 to 57, 9 to 57, 10 to 57, 11 to 57, 12 to 57, 13 to 57, 14 to 57, 15 to 57, 16 to 57, 17 to 57, 18 to 57, 19 to 57, 20 to 57, 21 to 57, 22 to 57, 23 to 57, 24 to 57, 25 to 57, 26 to 57, 27 to 57, 28 to 57, 29 to 57, 30 to 57, 31 to 57, 32 to 57, 33 to 57, 34 to 57, 35 to 57, 36 to 57, 37 to 57, 38 to 57, 39 to 57, 40 to 57, 41 to 57, 42 to 57, 43 to 57, 44 to 57, 45 to 57, 46 to 57, 47 to 57, 48 to 57, 49 to 57, 50 to 57, 51 to 57, 52 to 57, 53 to 57, 54 to 57, 55 to 57, 56 to 57, 0 to 56, 1 to 56, 2 to 56, 3 to 56, 4 to 56, 5 to 56, 6 to 56, 7 to 56, 8 to 56, 9 to 56, 10 to 56, 11 to 56, 12 to 56, 13 to 56, 14 to 56, 15 to 56, 16 to 56, 17 to 56, 18 to 56, 19 to 56, 20 to 56, 21 to 56, 22 to 56, 23 to 56, 24 to 56, 25 to 56, 26 to 56, 27 to 56, 28 to 56, 29 to 56, 30 to 56, 31 to 56, 32 to 56, 33 to 56, 34 to 56, 35 to 56, 36 to 56, 37 to 56, 38 to 56, 39 to 56, 40 to 56, 41 to 56, 42 to 56, 43 to 56, 44 to 56, 45 to 56, 46 to 56, 47 to 56, 48 to 56, 49 to 56, 50 to 56, 51 to 56, 52 to 56, 53 to 56, 54 to 56, 55 to 56, 0 to 55, 1 to 55, 2 to 55, 3 to 55, 4 to 55, 5 to 55, 6 to 55, 7 to 55, 8 to 55, 9 to 55, 10 to 55, 11 to 55, 12 to 55, 13 to 55, 14 to 55, 15 to 55, 16 to 55, 17 to 55, 18 to 55, 19 to 55, 20 to 55, 21 to 55, 22 to 55, 23 to 55, 24 to 55, 25 to 55, 26 to 55, 27 to 55, 28 to 55, 29 to 55, 30 to 55, 31 to 55, 32 to 55, 33 to 55, 34 to 55, 35 to 55, 36 to 55, 37 to 55, 38 to 55, 39 to 55, 40 to 55, 41 to 55, 42 to 55, 43 to 55, 44 to 55, 45 to 55, 46 to 55, 47 to 55, 48 to 55, 49 to 55, 50 to 55, 51 to 55, 52 to 55, 53 to 55, 54 to 55, 0 to 54, 1 to 54, 2 to 54, 3 to 54, 4 to 54, 5 to 54, 6 to 54, 7 to 54, 8 to 54, 9 to 54, 10 to 54, 11 to 54, 12 to 54, 13 to 54, 14 to 54, 15 to 54, 16 to 54, 17 to 54, 18 to 54, 19 to 54, 20 to 54, 21 to 54, 22 to 54, 23 to 54, 24 to 54, 25 to 54, 26 to 54, 27 to 54, 28 to 54, 29 to 54, 30 to 54, 31 to 54, 32 to 54, 33 to 54, 34 to 54, 35 to 54, 36 to 54, 37 to 54, 38 to 54, 39 to 54, 40 to 54, 41 to 54, 42 to 54, 43 to 54, 44 to 54, 45 to 54, 46 to 54, 47 to 54, 48 to 54, 49 to 54, 50 to 54, 51 to 54, 52 to 54, 53 to 54, 0 to 53, 1 to 53, 2 to 53, 3 to 53, 4 to 53, 5 to 53, 6 to 53, 7 to 53, 8 to 53, 9 to 53, 10 to 53, 11 to 53, 12 to 53, 13 to 53, 14 to 53, 15 to 53, 16 to 53, 17 to 53, 18 to 53, 19 to 53, 20 to 53, 21 to 53, 22 to 53, 23 to 53, 24 to 53, 25 to 53, 26 to 53, 27 to 53, 28 to 53, 29 to 53, 30 to 53, 31 to 53, 32 to 53, 33 to 53, 34 to 53, 35 to 53, 36 to 53, 37 to 53, 38 to 53, 39 to 53, 40 to 53, 41 to 53, 42 to 53, 43 to 53, 44 to 53, 45 to 53, 46 to 53, 47 to 53, 48 to 53, 49 to 53, 50 to 53, 51 to 53, 52 to 53, 0 to 52, 1 to 52, 2 to 52, 3 to 52, 4 to 52, 5 to 52, 6 to 52, 7 to 52, 8 to 52, 9 to 52, 10 to 52, 11 to 52, 12 to 52, 13 to 52, 14 to 52, 15 to 52, 16 to 52, 17 to 52, 18 to 52, 19 to 52, 20 to 52, 21 to 52, 22 to 52, 23 to 52, 24 to 52, 25 to 52, 26 to 52, 27 to 52, 28 to 52, 29 to 52, 30 to 52, 31 to 52, 32 to 52, 33 to 52, 34 to 52, 35 to 52, 36 to 52, 37 to 52, 38 to 52, 39 to 52, 40 to 52, 41 to 52, 42 to 52, 43 to 52, 44 to 52, 45 to 52, 46 to 52, 47 to 52, 48 to 52, 49 to 52, 50 to 52, 51 to 52, 0 to 51, 1 to 51, 2 to 51, 3 to 51, 4 to 51, 5 to 51, 6 to 51, 7 to 51, 8 to 51, 9 to 51, 10 to 51, 11 to 51, 12 to 51, 13 to 51, 14 to 51, 15 to 51, 16 to 51, 17 to 51, 18 to 51, 19 to 51, 20 to 51, 21 to 51, 22 to 51, 23 to 51, 24 to 51, 25 to 51, 26 to 51, 27 to 51, 28 to 51, 29 to 51, 30 to 51, 31 to 51, 32 to 51, 33 to 51, 34 to 51, 35 to 51, 36 to 51, 37 to 51, 38 to 51, 39 to 51, 40 to 51, 41 to 51, 42 to 51, 43 to 51, 44 to 51, 45 to 51, 46 to 51, 47 to 51, 48 to 51, 49 to 51, 50 to 51, 0 to 50, 1 to 50, 2 to 50, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 26 to 50, 27 to 50, 28 to 50, 29 to 50, 30 to 50, 31 to 50, 32 to 50, 33 to 50, 34 to 50, 35 to 50, 36 to 50, 37 to 50, 38 to 50, 39 to 50, 40 to 50, 41 to 50, 42 to 50, 43 to 50, 44 to 50, 45 to 50, 46 to 50, 47 to 50, 48 to 50, 49 to 50, 0 to 49, 1 to 49, 2 to 49, 3 to 49, 4 to 49, 5 to 49, 6 to 49, 7 to 49, 8 to 49, 9 to 49, 10 to 49, 11 to 49, 12 to 49, 13 to 49, 14 to 49, 15 to 49, 16 to 49, 17 to 49, 18 to 49, 19 to 49, 20 to 49, 21 to 49, 22 to 49, 23 to 49, 24 to 49, 25 to 49, 26 to 49, 27 to 49, 28 to 49, 29 to 49, 30 to 49, 31 to 49, 32 to 49, 33 to 49, 34 to 49, 35 to 49, 36 to 49, 37 to 49, 38 to 49, 39 to 49, 40 to 49, 41 to 49, 42 to 49, 43 to 49, 44 to 49, 45 to 49, 46 to 49, 47 to 49, 48 to 49, 0 to 48, 1 to 48, 2 to 48, 3 to 48, 4 to 48, 5 to 48, 6 to 48, 7 to 48, 8 to 48, 9 to 48, 10 to 48, 11 to 48, 12 to 48, 13 to 48, 14 to 48, 15 to 48, 16 to 48, 17 to 48, 18 to 48, 19 to 48, 20 to 48, 21 to 48, 22 to 48, 23 to 48, 24 to 48, 25 to 48, 26 to 48, 27 to 48, 28 to 48, 29 to 48, 30 to 48, 31 to 48, 32 to 48, 33 to 48, 34 to 48, 35 to 48, 36 to 48, 37 to 48, 38 to 48, 39 to 48, 40 to 48, 41 to 48, 42 to 48, 43 to 48, 44 to 48, 45 to 48, 46 to 48, 47 to 48, 0 to 47, 1 to 47, 2 to 47, 3 to 47, 4 to 47, 5 to 47, 6 to 47, 7 to 47, 8 to 47, 9 to 47, 10 to 47, 11 to 47, 12 to 47, 13 to 47, 14 to 47, 15 to 47, 16 to 47, 17 to 47, 18 to 47, 19 to 47, 20 to 47, 21 to 47, 22 to 47, 23 to 47, 24 to 47, 25 to 47, 26 to 47, 27 to 47, 28 to 47, 29 to 47, 30 to 47, 31 to 47, 32 to 47, 33 to 47, 34 to 47, 35 to 47, 36 to 47, 37 to 47, 38 to 47, 39 to 47, 40 to 47, 41 to 47, 42 to 47, 43 to 47, 44 to 47, 45 to 47, 46 to 47, 0 to 46, 1 to 46, 2 to 46, 3 to 46, 4 to 46, 5 to 46, 6 to 46, 7 to 46, 8 to 46, 9 to 46, 10 to 46, 11 to 46, 12 to 46, 13 to 46, 14 to 46, 15 to 46, 16 to 46, 17 to 46, 18 to 46, 19 to 46, 20 to 46, 21 to 46, 22 to 46, 23 to 46, 24 to 46, 25 to 46, 26 to 46, 27 to 46, 28 to 46, 29 to 46, 30 to 46, 31 to 46, 32 to 46, 33 to 46, 34 to 46, 35 to 46, 36 to 46, 37 to 46, 38 to 46, 39 to 46, 40 to 46, 41 to 46, 42 to 46, 43 to 46, 44 to 46, 45 to 46, 0 to 45, 1 to 45, 2 to 45, 3 to 45, 4 to 45, 5 to 45, 6 to 45, 7 to 45, 8 to 45, 9 to 45, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 26 to 45, 27 to 45, 28 to 45, 29 to 45, 30 to 45, 31 to 45, 32 to 45, 33 to 45, 34 to 45, 35 to 45, 36 to 45, 37 to 45, 38 to 45, 39 to 45, 40 to 45, 41 to 45, 42 to 45, 43 to 45, 44 to 45, 0 to 44, 1 to 44, 2 to 44, 3 to 44, 4 to 44, 5 to 44, 6 to 44, 7 to 44, 8 to 44, 9 to 44, 10 to 44, 11 to 44, 12 to 44, 13 to 44, 14 to 44, 15 to 44, 16 to 44, 17 to 44, 18 to 44, 19 to 44, 20 to 44, 21 to 44, 22 to 44, 23 to 44, 24 to 44, 25 to 44, 26 to 44, 27 to 44, 28 to 44, 29 to 44, 30 to 44, 31 to 44, 32 to 44, 33 to 44, 34 to 44, 35 to 44, 36 to 44, 37 to 44, 38 to 44, 39 to 44, 40 to 44, 41 to 44, 42 to 44, 43 to 44, 0 to 43, 1 to 43, 2 to 43, 3 to 43, 4 to 43, 5 to 43, 6 to 43, 7 to 43, 8 to 43, 9 to 43, 10 to 43, 11 to 43, 12 to 43, 13 to 43, 14 to 43, 15 to 43, 16 to 43, 17 to 43, 18 to 43, 19 to 43, 20 to 43, 21 to 43, 22 to 43, 23 to 43, 24 to 43, 25 to 43, 26 to 43, 27 to 43, 28 to 43, 29 to 43, 30 to 43, 31 to 43, 32 to 43, 33 to 43, 34 to 43, 35 to 43, 36 to 43, 37 to 43, 38 to 43, 39 to 43, 40 to 43, 41 to 43, 42 to 43, 0 to 42, 1 to 42, 2 to 42, 3 to 42, 4 to 42, 5 to 42, 6 to 42, 7 to 42, 8 to 42, 9 to 42, 10 to 42, 11 to 42, 12 to 42, 13 to 42, 14 to 42, 15 to 42, 16 to 42, 17 to 42, 18 to 42, 19 to 42, 20 to 42, 21 to 42, 22 to 42, 23 to 42, 24 to 42, 25 to 42, 26 to 42, 27 to 42, 28 to 42, 29 to 42, 30 to 42, 31 to 42, 32 to 42, 33 to 42, 34 to 42, 35 to 42, 36 to 42, 37 to 42, 38 to 42, 39 to 42, 40 to 42, 41 to 42, 0 to 41, 1 to 41, 2 to 41, 3 to 41, 4 to 41, 5 to 41, 6 to 41, 7 to 41, 8 to 41, 9 to 41, 10 to 41, 11 to 41, 12 to 41, 13 to 41, 14 to 41, 15 to 41, 16 to 41, 17 to 41, 18 to 41, 19 to 41, 20 to 41, 21 to 41, 22 to 41, 23 to 41, 24 to 41, 25 to 41, 26 to 41, 27 to 41, 28 to 41, 29 to 41, 30 to 41, 31 to 41, 32 to 41, 33 to 41, 34 to 41, 35 to 41, 36 to 41, 37 to 41, 38 to 41, 39 to 41, 40 to 41, 0 to 40, 1 to 40, 2 to 40, 3 to 40, 4 to 40, 5 to 40, 6 to 40, 7 to 40, 8 to 40, 9 to 40, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 26 to 40, 27 to 40, 28 to 40, 29 to 40, 30 to 40, 31 to 40, 32 to 40, 33 to 40, 34 to 40, 35 to 40, 36 to 40, 37 to 40, 38 to 40, 39 to 40, 0 to 39, 1 to 39, 2 to 39, 3 to 39, 4 to 39, 5 to 39, 6 to 39, 7 to 39, 8 to 39, 9 to 39, 10 to 39, 11 to 39, 12 to 39, 13 to 39, 14 to 39, 15 to 39, 16 to 39, 17 to 39, 18 to 39, 19 to 39, 20 to 39, 21 to 39, 22 to 39, 23 to 39, 24 to 39, 25 to 39, 26 to 39, 27 to 39, 28 to 39, 29 to 39, 30 to 39, 31 to 39, 32 to 39, 33 to 39, 34 to 39, 35 to 39, 36 to 39, 37 to 39, 38 to 39, 0 to 38, 1 to 38, 2 to 38, 3 to 38, 4 to 38, 5 to 38, 6 to 38, 7 to 38, 8 to 38, 9 to 38, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 26 to 38, 27 to 38, 28 to 38, 29 to 38, 30 to 38, 31 to 38, 32 to 38, 33 to 38, 34 to 38, 35 to 38, 36 to 38, 37 to 38, 0 to 37, 1 to 37, 2 to 37, 3 to 37, 4 to 37, 5 to 37, 6 to 37, 7 to 37, 8 to 37, 9 to 37, 10 to 37, 11 to 37, 12 to 37, 13 to 37, 14 to 37, 15 to 37, 16 to 37, 17 to 37, 18 to 37, 19 to 37, 20 to 37, 21 to 37, 22 to 37, 23 to 37, 24 to 37, 25 to 37, 26 to 37, 27 to 37, 28 to 37, 29 to 37, 30 to 37, 31 to 37, 32 to 37, 33 to 37, 34 to 37, 35 to 37, 36 to 37, 0 to 36, 1 to 36, 2 to 36, 3 to 36, 4 to 36, 5 to 36, 6 to 36, 7 to 36, 8 to 36, 9 to 36, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 26 to 36, 27 to 36, 28 to 36, 29 to 36, 30 to 36, 31 to 36, 32 to 36, 33 to 36, 34 to 36, 35 to 36, 0 to 35, 1 to 35, 2 to 35, 3 to 35, 4 to 35, 5 to 35, 6 to 35, 7 to 35, 8 to 35, 9 to 35, 10 to 35, 11 to 35, 12 to 35, 13 to 35, 14 to 35, 15 to 35, 16 to 35, 17 to 35, 18 to 35, 19 to 35, 20 to 35, 21 to 35, 22 to 35, 23 to 35, 24 to 35, 25 to 35, 26 to 35, 27 to 35, 28 to 35, 29 to 35, 30 to 35, 31 to 35, 32 to 35, 33 to 35, 34 to 35, 0 to 34, 1 to 34, 2 to 34, 3 to 34, 4 to 34, 5 to 34, 6 to 34, 7 to 34, 8 to 34, 9 to 34, 10 to 34, 11 to 34, 12 to 34, 13 to 34, 14 to 34, 15 to 34, 16 to 34, 17 to 34, 18 to 34, 19 to 34, 20 to 34, 21 to 34, 22 to 34, 23 to 34, 24 to 34, 25 to 34, 26 to 34, 27 to 34, 28 to 34, 29 to 34, 30 to 34, 31 to 34, 32 to 34, 33 to 34, 0 to 33, 1 to 33, 2 to 33, 3 to 33, 4 to 33, 5 to 33, 6 to 33, 7 to 33, 8 to 33, 9 to 33, 10 to 33, 11 to 33, 12 to 33, 13 to 33, 14 to 33, 15 to 33, 16 to 33, 17 to 33, 18 to 33, 19 to 33, 20 to 33, 21 to 33, 22 to 33, 23 to 33, 24 to 33, 25 to 33, 26 to 33, 27 to 33, 28 to 33, 29 to 33, 30 to 33, 31 to 33, 32 to 33, 0 to 32, 1 to 32, 2 to 32, 3 to 32, 4 to 32, 5 to 32, 6 to 32, 7 to 32, 8 to 32, 9 to 32, 10 to 32, 11 to 32, 12 to 32, 13 to 32, 14 to 32, 15 to 32, 16 to 32, 17 to 32, 18 to 32, 19 to 32, 20 to 32, 21 to 32, 22 to 32, 23 to 32, 24 to 32, 25 to 32, 26 to 32, 27 to 32, 28 to 32, 29 to 32, 30 to 32, 31 to 32, 0 to 31, 1 to 31, 2 to 31, 3 to 31, 4 to 31, 5 to 31, 6 to 31, 7 to 31, 8 to 31, 9 to 31, 10 to 31, 11 to 31, 12 to 31, 13 to 31, 14 to 31, 15 to 31, 16 to 31, 17 to 31, 18 to 31, 19 to 31, 20 to 31, 21 to 31, 22 to 31, 23 to 31, 24 to 31, 25 to 31, 26 to 31, 27 to 31, 28 to 31, 29 to 31, 30 to 31, 0 to 30, 1 to 30, 2 to 30, 3 to 30, 4 to 30, 5 to 30, 6 to 30, 7 to 30, 8 to 30, 9 to 30, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 26 to 30, 27 to 30, 28 to 30, 29 to 30, 0 to 29, 1 to 29, 2 to 29, 3 to 29, 4 to 29, 5 to 29, 6 to 29, 7 to 29, 8 to 29, 9 to 29, 10 to 29, 11 to 29, 12 to 29, 13 to 29, 14 to 29, 15 to 29, 16 to 29, 17 to 29, 18 to 29, 19 to 29, 20 to 29, 21 to 29, 22 to 29, 23 to 29, 24 to 29, 25 to 29, 26 to 29, 27 to 29, 28 to 29, 0 to 28, 1 to 28, 2 to 28, 3 to 28, 4 to 28, 5 to 28, 6 to 28, 7 to 28, 8 to 28, 9 to 28, 10 to 28, 11 to 28, 12 to 28, 13 to 28, 14 to 28, 15 to 28, 16 to 28, 17 to 28, 18 to 28, 19 to 28, 20 to 28, 21 to 28, 22 to 28, 23 to 28, 24 to 28, 25 to 28, 26 to 28, 27 to 28, 0 to 27, 1 to 27, 2 to 27, 3 to 27, 4 to 27, 5 to 27, 6 to 27, 7 to 27, 8 to 27, 9 to 27, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 26 to 27, 0 to 26, 1 to 26, 2 to 26, 3 to 26, 4 to 26, 5 to 26, 6 to 26, 7 to 26, 8 to 26, 9 to 26, 10 to 26, 11 to 26, 12 to 26, 13 to 26, 14 to 26, 15 to 26, 16 to 26, 17 to 26, 18 to 26, 19 to 26, 20 to 26, 21 to 26, 22 to 26, 23 to 26, 24 to 26, 25 to 26, 0 to 25, 1 to 25, 2 to 25, 3 to 25, 4 to 25, 5 to 25, 6 to 25, 7 to 25, 8 to 25, 9 to 25, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, 24 to 25, 0 to 24, 1 to 24, 2 to 24, 3 to 24, 4 to 24, 5 to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 0 to 23, 1 to 23, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, 22 to 23, 0 to 22, 1 to 22, 2 to 22, 3 to 22, 4 to 22, 5 to 22, 6 to 22, 7 to 22, 8 to 22, 9 to 22, 10 to 22, 11 to 22, 12 to 22, 13 to 22, 14 to 22, 15 to 22, 16 to 22, 17 to 22, 18 to 22, 19 to 22, 20 to 22, 21 to 22, 0 to 21, 1 to 21, 2 to 21, 3 to 21, 4 to 21, 5 to 21, 6 to 21, 7 to 21, 8 to 21, 9 to 21, 10 to 21, 11 to 21, 12 to 21, 13 to 21, 14 to 21, 15 to 21, 16 to 21, 17 to 21, 18 to 21, 19 to 21, 20 to 21, 0 to 20, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, 19 to 20, 0 to 19, 1 to 19, 2 to 19, 3 to 19, 4 to 19, 5 to 19, 6 to 19, 7 to 19, 8 to 19, 9 to 19, 10 to 19, 11 to 19, 12 to 19, 13 to 19, 14 to 19, 15 to 19, 16 to 19, 17 to 19, 18 to 19, 0 to 18, 1 to 18, 2 to 18, 3 to 18, 4 to 18, 5 to 18, 6 to 18, 7 to 18, 8 to 18, 9 to 18, 10 to 18, 11 to 18, 12 to 18, 13 to 18, 14 to 18, 15 to 18, 16 to 18, 17 to 18, 0 to 17, 1 to 17, 2 to 17, 3 to 17, 4 to 17, 5 to 17, 6 to 17, 7 to 17, 8 to 17, 9 to 17, 10 to 17, 11 to 17, 12 to 17, 13 to 17, 14 to 17, 15 to 17, 16 to 17, 0 to 16, 1 to 16, 2 to 16, 3 to 16, 4 to 16, 5 to 16, 6 to 16, 7 to 16, 8 to 16, 9 to 16, 10 to 16, 11 to 16, 12 to 16, 13 to 16, 14 to 16, 15 to 16, 0 to 15, 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15, 0 to 14, 1 to 14, 2 to 14, 3 to 14, 4 to 14, 5 to 14, 6 to 14, 7 to 14, 8 to 14, 9 to 14, 10 to 14, 11 to 14, 12 to 14, 13 to 14, 0 to 13, 1 to 13, 2 to 13, 3 to 13, 4 to 13, 5 to 13, 6 to 13, 7 to 13, 8 to 13, 9 to 13, 10 to 13, 11 to 13, 12 to 13, 0 to 12, 1 to 12, 2 to 12, 3 to 12, 4 to 12, 5 to 12, 6 to 12, 7 to 12, 8 to 12, 9 to 12, 10 to 12, 11 to 12, 0 to 11, 1 to 11, 2 to 1, 3 to 1, 4 to 1, 5 to 1, 6 to 1, 7 to 1, 8 to 1, 9 to 11, 10 to 1, 0 to 10, 1 to 10, 2 to 10, 3 to 10, 4 to 10, 5 to 10, 6 to 10, 7 to 10, 8 to 10, 9 to 10, 0 to 9, 1 to 9, 2 to 9, 3 to 9, 4 to 9, 5 to 9, 6 to 9, 7 to 9, 8 to 9, 0 to 8, 1 to 8, 2 to 8, 3 to 8, 4 to 8, 5 to 8, 6 to 8, 7 to 8, 0 to 7, 1 to 7, 2 to 7, 3 to 7, 4 to 7, 5 to 7, 6 to 7, 0 to 6, 1 to 6, 2 to 6, 3 to 6, 4 to 6, 5 to 6, 0 to 5, 1 to 5, 2 to 5, 3 to 5, 4 to 5, 0 to 4, 1 to 4, 2 to 4, 3 to 4, 0 to 3, 1 to 3, 2 to 3, 0 to 2, 1 to 2, or 0 to 1 weight % water.

The acetyl feed stream 15 can be mixed with liquid or vaporous water, i.e., steam, so that the feed stream comprises about 40 to about 99 weight % acetic acid, up to about 50 weight of the impurity, and optionally up to about 30 weight % water, based on the total weight of the feed stream 15.

The acetyl feed stream 15 may contain up to about 10 weight percent acetic anhydride, or about 0.5 to about 10 weight percent acetic anhydride, or about 1.0 to about 10 weight percent acetic anhydride, or about 2.0 to about 10 weight percent acetic anhydride, or about 3.0 to about 10 weight percent acetic anhydride, or about 4.0 to about 10 weight percent acetic anhydride, or about 5.0 to about 10 weight percent acetic anhydride, or about 6.0 to about 10 weight percent acetic anhydride, or about 7.0 to about 10 weight percent acetic anhydride, or about 8.0 to about 10 weight percent acetic anhydride. It is understood that such ranges include 0.5 to 9, 1 to 9, 2 to 9, 3 to 9, 4 to 9, 5 to 9, 6 to 9, 7 to 9, 8 to 9, 0.5 to 8, 1 to 8, 2 to 8, 3 to 8, 4 to 8, 5 to 8, 6 to 8, 7 to 8, 0.5 to 7, 1 to 7, 2 to 7, 3 to 7, 4 to 7, 5 to 7, 6 to 7, 0.5 to 6, 1 to 6, 2 to 6, 3 to 6, 4 to 6, 5 to 6, 0.5 to 5, 1 to 5, 2 to 5, 3 to 5, 4 to 5, 0.5 to 4, 1 to 4, 2 to 4, 3 to 4, 0.5 to 3, 1 to 3, 2 to 3, 0.5 to 2, 1 to 2, and 0.5 to 1 weight % acetic anhydride wherein the weight percentage is based on the total constituents of the feed stream 15.

Optionally, acetic acid may also be added to the wet acetic acid feed stream to bring the final concentration of acetic acid to one of the aforementioned ranges. It is to be understood that the above delineated weight percentages are based on the total weight of all the constituents present in the acetyl feed stream 15. It is also to be understood that the ranges specified include all concentrations, weight percentages and ranges in between the ranges specified and that such ranges have been specified as whole numbers for sake of brevity.

In the vaporization unit 20 typically 75 to 99 weight %, of the acetyl feed 15 is vaporized by boiling against steam, to produce vaporized acid stream 25. The acid feed stream 15 is vaporized at typically 110° C.-195° C. and at a pressure of from 0.7 to 7.0 bar, or from about 115° C.-160° C., and at a pressure of from 0.9 to 3.2 bar. Typically, 1.0 weight % to 25.0 weight % of the incoming wet acid stream 15 may be removed as sludge stream 30 from the vaporizer 20 to prevent fouling of the vaporizer equipment, the furnace superheater 45, and the catalyst bed, as well as to remove non-volatile components such as salts and tars. The vaporizer 20 can be any apparatus known to persons skilled in the art such as, for example, kettle-type, thermosyphon-type, wiped-film, falling film, and thin film evaporators. Optionally, steam stream 35 may be added to the vaporized acid to bring the water concentration in the wet acid stream 40 from about 5 weight % to about 70 weight % water, or from about 10 weight % to about 20 weight % water, based on the total weight of the wet acid stream 40. This water addition helps mitigate coke formation in the ketonization reactor and increases the yield of acetone from acetic acid.

Wet acid stream 40 is further superheated to the desired reaction inlet temperature in a superheater or furnace 45 to produce superheated feed stream 50. The term “superheated,” as used herein, is intended to have the commonly understood meaning of a vapor heated to a temperature above its dew point at a given pressure. The temperature of the superheated feed stream 50 can be about 350° C. to about 650° C., or from about 350° C. to about 600° C., or from about 350° C. to about 550° C., or from about 300° C. to about 450° C. Typically, the wet acid feed stream 40 is preheated to the desired inlet temperature in a furnace and then passed through the ketonization catalyst bed.

The vaporized feed mixture 40 may be conveyed through the superheater 45 using a multi-pass tubular configuration inside of an insulated furnace box. If a direct fired furnace is used, then heat is provided to the furnace by combustion of fuel 52 with air stream 53, and diluted for temperature control by at least a portion of a by-product carbon dioxide stream 54 by conduit 55. The fuel for the furnace may be any combustible material of sufficient energy density, including, but not limited to natural gas, propane, butane, natural gas liquids, liquefied petroleum gases, hydrogen, refinery off gases, pyrolysis gasoline, ethanol, methanol, heavy organic by-products from the ketonization reactor, such as mesityl oxide and related compounds, the sludge stream 30 from the acid vaporizer 20, or petroleum fractions, such as gasoline, kerosene, bunker fuel, heating oil, and the like. Design of the burners is highly dependent on the fuel chosen as is well known to those skilled in the art. Natural gas is the preferred furnace fuel.

Heat may be transferred to the tubes containing the wet acid feed 40 via radiated and convective heat transfer mechanisms. In order to prevent high tube skin temperatures which tend to lead to coking on the process fluid side of the tubes, it is desirable that the primary heat transfer occur in a portion of the feed superheater 45 where sufficient diluent has been added to the already combusted hot fuel/air mixture to lower its temperature to 700° C. to 900° C. in a post combustion zone. The diluent gas may be air or by-product carbon dixode stream or a combinations thereof. The preferred diluent, above the excess air required for combustion, is the by-product carbon dioxide stream 54. Although any conventional source of oxygen can be used, air is generally the least expensive and most readily available source of oxygen. Such furnace configurations are described in greater detail in U.S. Pat. No. 8,779,208, the entire disclosure of which is incorporated herein by reference.

Air feed to the combustion zone of the furnace 45 may be by natural or forced draft. Sufficient air is supplied to give 10 to 40% excess oxygen over the stoichiometric amount required for complete combustion of both the fuel and the VOC components in the by-product carbon dioxide stream. If the by-product carbon dioxide stream is utilized for combustion, then, desirably, residence time in the post combustion zone for the oxidative destruction of the VOC's can be from 0.02 to 5.0 seconds, or from 0.1 to 0.5 seconds. The temperature in the post combustion zone of the furnace where VOC destruction takes place can be from 600° C.-900° C., or from 650° C.-800° C. The furnace is designed such that residence time and temperature are sufficient for at least 50%, or at least 65% of the total VOC's present originally in the by-product carbon dioxide stream are oxidatively destroyed.

The super heater furnace 45 is sized to supply sufficient heat to raise the wet acid feed 50 to proper reaction temperature, providing both sensible heat and sufficient thermal energy to compensate for the endothermic heat of ketonization. Typically the furnace will be designed to supply 0.7 to 2.6 million J/kg, more typically 0.75-0.9 million J/kg of acetic acid fed, depending on water content of the vaporized acid stream.

When run in adiabatic mode, the wet acid feed 15 is preheated to the desired reactor inlet temperature, typically 350° C. to 650° C., or from 350° C. to 500° C. in a direct-fired furnace or superheater 45 in order to supply the heat of reaction. As discussed above, it is common for the wet acid feed 15 to be conveyed through the superheater 45 using a multi-pass tubular configuration situated in an insulated furnace box wherein a fuel is combusted with oxygen and diluent to generate high temperature heat.

The superheated feed stream 50 coming from the superheater 45 is passed to the ketonization reactor 60 where the acetic acid and other reactive feed molecules, if present, are converted over a heterogeneous ketonization catalyst to produce a crude product mixture 65 comprising acetone, water, carbon dioxide, unreacted acetic acid, acetic acid azeotrope-forming compounds, and other minor by-products. The ketonization reactor 60 can be any reactor format known in the art to be suitable for gas-phase endothermic reactions. For example, the ketonization reaction may be conducted using a fixed, fluidized, or moving bed reactor. The ketonization reaction can be carried out in a single stage adiabatic fixed bed reactor; a multiple-stage adiabatic fixed bed reactor with interstage heating or hot-shotting; or a tubular fixed bed reactor in a fired furnace or molten salt heating bath.

Typically, about 90 mole % to about 100 mole % of the acetic acid will be converted to acetone, carbon dioxide, and water in a single stage adiabatic reactor. The inlet pressure to the ketonization reactor can be from about 0.5 bars to about 10 bars absolute. The temperature range for the ketonization reactor can be about 300° C. to about 600° C. over the length of the reactor. Preferably, the reaction is carried out in the vapor phase at elevated temperatures under the following conditions. The reaction temperature may be at least 300° C., or at least 325° C., or at least 350° C. In terms of ranges, the reaction temperature may range from 300° C. to 550° C., or from 325° C. to 500° C., or from 350° C. to 450° C. The pressure may range from 0.5 bars to about 10 bars absolute, or from 0.5 bars to about 8 bars absolute, or from 0.9 to about 7 bars absolute, or from 1.1 to about 5 bars absolute. The reactants may be fed to the reactor 60 at a gas hourly space velocity (GHSV) greater than 500 hr.-1, or greater than 1000 hr.-1, or greater than 2500 hr.-1 or even greater than 5000 hr.-1. In terms of ranges the GHSV may range from 50 hr.-1 to 50,000 hr.-1, or from 500 hr.-1 to 30,000 hr.-1, or from 1000 hr.-1 to 10,000 hr.-1, or from 1000 hr.-1 to 6500 hr.-1. When run in single stage, adiabatic mode, the reactor temperature will be highest at the inlet and drop to the lowest value at the outlet because of the endothermic heat of reaction. The temperature drop across the reactor can be as much as from about 40° to about 75° C., depending on water content of the feed and conversion of acetic acid.

Contact or residence time can vary widely, depending upon such variables as amount of acetic acid, catalyst, reactor, temperature, and pressure. Typical contact times range from a fraction of a second to more than several hours when a catalyst system other than a fixed bed is used, with preferred contact times from 0.1 to 100 seconds, or from 0.3 to 80 seconds, or from 0.4 to 30 seconds. One skilled in the art will understand that the contact or residence time can vary due to many factors present in ketonization reactor such as pressure, temperature, catalyst activity, catalyst selectivity, flow through, and the like. Accordingly, adjustment of the residence time to obtain the level of conversion of acetyl to ketone, such as acetic acid to acetone, is well within the understanding of one skilled in the art.

In the ketonization reactor, the superheated feed mixture 50 contacts a metal oxide catalyst where the acetic acid and other reactive species, such as trace amounts of propionic acid or acetic anhydride, are converted into a gaseous crude product mixture 65 comprising acetone, other ketones, water, the impurity having at least one acetic acid azeotrope-forming compound, and byproducts from the ketonization reaction. Such byproducts include, for example, carbon dioxide, and one or more volatile organic compounds having a boiling point less than or equal to 250° C. measured at a standard atmospheric pressure of 1 bar absolute. Some examples of volatile organic compounds include, but are not limited to, methane, ethane, acetone, methyl acetate, isobutylene, mesityl oxide, terpenes, methyl ethyl ketone, and other low molecular weight aldehydes, ketones, hydrocarbons, olefins, alcohols, and esters. The crude product mixture 65 comprises from 25 to about 70 weight % acetone, about 25 to about 75 weight % water, and about 10 ppm to about 25 weight % of the impurity, wherein the weight % is based on the total weight of the constituents of the product mixture 65 and absent any catalyst carryover; or from 40 to about 70 weight % acetone, about 30 to about 60 weight % water, and about 100 ppm to about 20 weight % of the impurity; or from 50 to about 70 weight % acetone, about 30 to about 35 weight % water, and about 200 ppm to about 15 weight % of the impurity.

Optionally, the vaporized acetic acid may be fed to the ketonization reactor along with a carrier gas. The acetic acid is transferred to the vapor state by passing a carrier gas through the acetic acid at a temperature at or below 150° C., followed by heating of the gaseous stream to the reactor inlet temperature. In the case where a carrier gas is utilized, it may be selected from such gases as hydrogen, nitrogen, argon, helium, carbon dioxide or combinations thereof. Although the carrier gas may be inert, it is also contemplated that hydrogen can be used which may also reduce the acetic acid.

The ketonization reactor may be operated in isothermal mode, wherein the ketonization catalyst is charged to tubes placed in a furnace box and reaction occurs simultaneously with direct-fired heating.

The metal oxide catalyst(s) utilized in the ketonization reaction of the present invention include oxides rare earth metals, transition metals, alkali metals, and alkaline earth metals, either alone or in combination with one or more metals. The metal oxide catalysts can exhibit both acid and base functionalities. The metal oxides may be employed either alone or in combination with one or more metals. Representative examples of metal oxide ketonization catalysts may be found in Glinski et al, “Ketones from Monocarboxylic Acids: Catalytic Ketonization Over Oxide Catalysts”, Applied Catalysis A: General, Vol. 128, (1995) pp. 209-217. The metal oxides may be supported on inorganic carriers well-known to persons skilled in the art such as, for example, silica, titania, or alumina. The activity and selectivity of the metal oxide catalyst may be enhanced by the presence of metal oxides of the Group IA metals, such as lithium, sodium, potassium, and cesium as disclosed, for example, by U.S. Pat. No. 4,950,763.

For producing acetone, the type of support influences the conversion of acetic acid and selectivity to acetone. Some specific examples of metal oxide ketonization catalysts include, but are not limited to, oxides of cerium, thorium, lanthanum, manganese, zirconium, titanium, zinc, chromium, lead, iron, niobium, molybdenum, bismuth, cadmium, copper, nickel, magnesium, aluminum, and mixtures thereof. For example, the superheated feed stream has a temperature of about 300° C. to about 600° C. and the metal oxide catalyst comprises an oxide of titanium, zirconium, thorium, cerium, lanthanum, or a mixture thereof. The support can be present in an amount from 50 weight % to 99.5 weight %, or from 75 weight % to 99 weight %, or from 80 weight % to 90 weight %, based on the weight of the catalyst.

The metal oxide catalyst may be further impregnated with about 0.05 to about 50 weight percent, or about 1 weight percent to about 25 weight percent, or about 10 weight percent to about 20 weight percent, based on the total weight of the catalyst, of lithium, sodium, potassium, cesium, lanthanum, cerium, or a combination thereof. Alternatively, the ketonization catalyst can be impregnated with about 0.05 to about 50 weight percent, or about 1 weight percent to about 25 weight percent, or about 10 weight percent to about 20 weight percent, based on the total weight of the catalyst, of lithium, sodium, potassium, cesium, or a mixture thereof. It is also within the inventive concept for the ketonization catalyst to comprise titanium dioxide impregnated with about 1 to about 10 weight percent, based on the weight of the catalyst, with at least one of lithium, sodium, cesium, or potassium. The metal loading may vary depending on the type of active metal. The titanium dioxide can be in the anatase form.

The surface area of the ketonization catalyst can range from about 10 to about 400 m2/g of catalyst. Other examples of catalyst surface areas are about 20 to about 200 m2/g and about 50 to about 200 m2/g. The impregnated and/or supported catalysts can be prepared in accordance with methods well-known to persons skilled in the art such as, for example, by thoroughly mixing metal salt solutions of the catalyst and optional catalyst promoter with the carrier or support material. Capillary action then draws the precursor into the pores in the support. The catalyst is then dried and calcined. The catalyst may be in any of the commonly used catalyst shapes such as, for example, spheres, granules, pellets, chips, rings, extrudates, or powders that are well-known in the art. The ketonization catalyst can be regenerated by heating in the presence of an oxygen-containing gas at a temperature of about 375° C. to about 550° C.

The crude product mixture or gaseous reactor effluent 65, is cooled and separated in recovery zone 70 to produce a gaseous, non-condensable, by-product carbon dioxide stream 54 and a liquid crude acetone stream 75. The by-product carbon dioxide stream 54 comprises non-condensable compounds such as carbon dioxide, isobutylene, methane, hydrogen, other minor VOC's, and traces of acetone and higher by-products. The by-product carbon dioxide stream 54 may be sent in its entirety through conduit 55 to furnace 45, or a portion emitted directly through conduit 77 for proper disposal. Typically, during normal operation all of stream 54 will be sent to furnace 45 for combustion of VOC's, although at start up, or during furnace up-sets, a fraction or all of stream 54 may exit the process by stream 77 without further treatment. The liquid crude acetone stream 75 comprises the majority of the acetone, water, impurity and heavy by-products.

In the recovery zone 70 the ketone component can be separated from the carbon dioxide, carrier gas, if utilized, and one or more ketonization byproducts by conventional methods known to persons skilled in the art. For example, as illustrated by the ketonization of acetic acid to acetone, the gaseous product mixture from the ketonization reactor can be separated by direct condensation or absorption of the gaseous ketonization reactor product mixture into water or other solvent to produce a condensed crude acetone stream and a vaporous non-condensable byproduct stream comprising carbon dioxide and the byproducts such as isobutylene, hydrogen, methane, and higher ketones.

The separation step comprises cooling the gaseous product mixture by contact with a heat exchanger or a solvent. For example, the ketone component, i.e. acetone, may be condensed by indirect cooling in a heat exchanger against water, chilled brine, chilled glycol or the like, or via direct contact cooling with an injected solvent, such as water. After cooling, phase separation produces a vapor byproduct stream comprising the majority of the non-condensable components (such as, carbon dioxide, methane, isobutylene, and hydrogen), along with small amounts of acetone and higher boiling impurities; and a liquid crude acetone stream comprising the majority of the acetone, water, heavy byproducts from the reactor and the impurity having at least one acetic acid azeotrope-forming compound. The temperature range of the condenser operation is 0 to about 40° C., or from about 5° C. to 25° C. The condensed effluent from the ketonization reaction, comprises about 25 to about 70 weight percent acetone, about 25 to about 75 weight percent water, and about 10 ppm to about 25 weight percent of the acetic acid azeotrope-forming impurities and may further include about 0.1 to about 2 weight percent mesityl oxide. Generally recovery of acetone by condensation results in about 90% recovery of the acetone, or greater than 95% of the acetone is recovered, or greater than about 99% of the acetone is recovered.

High recovery of acetone by condensation alone, however, requires very low temperatures because of the volatile nature of acetone and the large volume of the non-condensable carbon dioxide present in the gaseous reactor effluent. The invention also includes recovering the acetone from the gaseous reactor effluent by absorption into a solvent such as, for example, water. Generally, the recovery of acetone by countercurrent absorption into water results in about 99 mole %, or about 99.5 mole %, or about 99.8 mole % recovery of acetone, based on the acetone fed to the absorber. The absorption may be carried out by any means known to those skilled in the art, for example, by contacting the gaseous crude product mixture with water in a countercurrent absorber such as, for example, a packed or trayed absorption tower. The gaseous crude product mixture containing acetone can be fed to the bottom of the absorption tower and acetone-lean solvent, e.g., water, can be fed to the top of the tower, which permits the gas and liquid phases co-mingle in a countercurrent flow pattern. The gaseous crude absorber stream comprises a vaporous acetone-lean carbon dioxide stream that is removed from the top of the tower or absorber, and the liquid crude absorber product stream comprises an acetone-rich stream which is removed from the bottom of the column. The gaseous crude absorber stream comprises less than about 50 mole % of the acetone in the crude product mixture coming from the ketonization reactor, and the liquid crude absorber stream comprises greater than about 50 mole % of the acetone in the crude product mixture coming from the ketonization reactor, or the liquid crude absorber stream comprises greater than about 70 mole % of the acetone in the crude product mixture coming from the ketonization reactor, or the liquid crude absorber stream comprises greater than about 90 mole % of the acetone in the crude product mixture coming from the ketonization reactor.

The solvent-to-feed weight ratio is typically about 0.5:1 to about 3:1. The high heat of absorption of acetone, however, may require heat removal to minimize solvent flow, staging, and to enable the maximum recovery of acetone. For example, the heat of absorption may be removed by side draw coolers or by a heat-exchanged pump around loop in which liquid from the bottom effluent of the absorber is pumped through a heat exchanger and fed back into the column, typically about one-quarter to about one-half of the distance from the bottom of the column to the top. The flow in the pump around loop may be about 0.5 to about 10 times the flow of the crude acetone product removed from the bottom of the absorber, or about 1 to about 4 times the flow of the crude acetone product. For example, the temperature range of absorber operation can be about 10° to about 65° C., or about 25° to about 50° C.

Any solvent with a suitable partition coefficient for acetone can be used in the absorber. Some representative examples of absorber solvents include, but are not limited to, water, C5 to C20 ketones, C2 to C16 carboxylic acids, C6 to C12 hydrocarbons, C6 to C16 ethers, C5 to C12 esters, and C3 to C12 alcohols. Some specific examples of absorber solvents are 2-pentanone, 4-methyl-2-pentaone, 2-heptanone, 5-methyl-2-hexanone, 4-heptanone, 2,4-dimethyl-5-pentanone, 2,5-dimethyl-4-heptanone, acetic acid, propionic acid, i-butyric acid, n-butyric acid, i-valeric acid, n-valeric acid, n-hexanoic acid, 2-ethyl-hexanoic acid, toluene, benzene, o-/m-/p-xylenes, diisopropyl ether, dipropylether, tertiary amyl methyl ether, dibutyl ether, n-propyl acetate, i-propyl acetate, n-butyl acetate, i-butyl acetate, sec-butyl acetate, 2-ethylhexyl acetate, ethyl propionate, n-propyl propionate, isopropyl propionate, ethyl n-butyrate, ethyl i-butyrate, methyl 2-ethylhexanoate, isopropanol, n-propanol, sec-butanol, i-butanol, n-butanol, n-hexanol, 2-ethylhexanol, and n-decanol.

With either condensation or absorption operations in the recovery zone, the non-condensable by-product stream will generally comprise about 95 to about 99.9 mole % carbon dioxide, 0 to about 0.4 mole percent methane, 0 to about 0.5 mole percent hydrogen, and about 0.02 to about 0.8 mole percent isobutylene on an acetone and water free basis. Additionally, the carbon dioxide by-product stream may contain unrecovered acetone, typically 0.05 to 5 mole percent acetone, water, 0.1 to 4 mole percent, and 0 to 100 ppm levels of other heavier by-products, based on the total weight of the carbon dioxide by-product stream.

The liquid crude acetone stream 75 obtained after condensation or absorption, i.e., the distillation feed, can comprise about 25 to about 70 weight percent acetone, about 25 to about 75 weight percent water, about 0.05 to about 2 weight % acetic acid, about 0.5 to about 2 weight percent mesityl oxide and other related by-products such as, for example, isophorone and mesitylene, and from about 0.05 weight % to about 25 weight percent of the impurity comprising at least one acetic acid azeotrope-forming compound, wherein the weight % is based on the total weight of the crude liquid acetone stream 75. The liquid crude acetone stream 75 may also comprise about 25 to about 85 weight percent acetone, about 15 to about 75 weight percent water, about 0.05 to about 2 weight % acetic acid, about 0.5 to about 2 weight percent mesityl oxide and other related by-products such as, for example, isophorone and mesitylene, and from about 0.1 weight % to about 20 weight percent of the impurity comprising at least one acetic acid azeotrope-forming compound; or the liquid crude acetone stream 75 can comprise about 25 to about 95 weight percent acetone, about 5 to about 75 weight percent water, about 0.05 to about 2 weight % acetic acid, about 0.5 to about 2 weight percent mesityl oxide and other related by-products such as, for example, isophorone and mesitylene, and from about 0.7 weight % to about 20 weight percent of the impurity comprising at least one acetic acid azeotrope-forming compound. It is to be understood that the aforementioned weight percentages are based on the weight of the liquid crude acetone stream and the sum of the constituent weight percentages equals 100%.

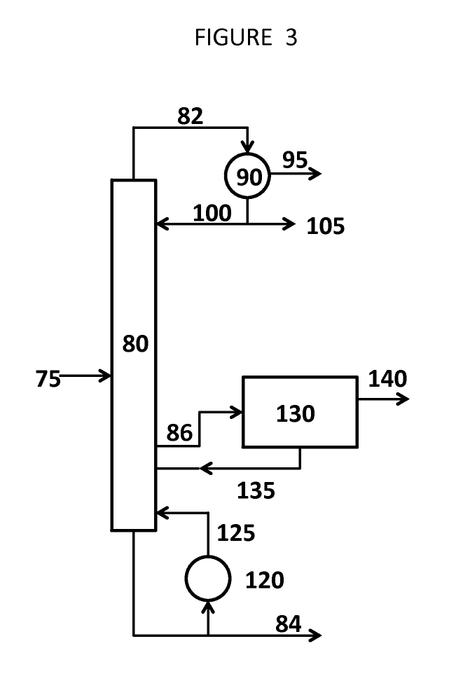

Referring to FIG. 3, the distillation step is described in greater detail. The liquid crude acetone stream 75 is introduced into the distillation column 80. The lower boiling constituents in the liquid crude acetone stream 75 travel up the distillation column 80 and are removed as a lower boiling fraction stream 82, and are at least partially condensed in condenser 90. Condenser 90 may be run as a total or partial condenser as is well known in the art. Generally, condenser 90 acts as a partial condenser for removal from the system light gases 95 originally dissolved in the liquid crude acetone stream 75. Non-limiting examples of such light gases include carbon dioxide, isobutylene, methane, and hydrogen. The partial condenser 90 cools lower boiling fraction stream 82 to 10° C. to about 50° C., or from about 25° C. to 45° C. A portion of the condensed vapors is returned to the distillation column 80 as reflux 100 and the remainder is withdrawn as the distillate product 105. The reflux ratio, (defined as the mass of stream 100/the mass of stream 105), depends on the desired purity of the product acetone distillate, but generally is from about 0.5 to 8, or from about 0.6 to 2.

Distillate stream 105, i.e., the lower boiling fraction, comprises greater than about 95 weight % acetone and a minor amount of the water and the acetic acid azeotrope-forming compound, based on the weight of the constituents in the distillate stream 105; or from about 95 to about 99 weight percent acetone, about 0.1 to about 5 weight percent water and from about 50 ppm to about 1.0 weight percent of the impurity having at least one acetic acid azeotrope-forming compound, based on the weight of the constituents in the distillate stream 105, or from about 95 to about 99 weight percent acetone, about 0.1 to about 2 weight percent water and from about 500 ppm to about 1 weight % of the impurity comprising at least one acetic acid azeotrope-forming compounds such as methanol, methyl isobutyl ketone, mesityl oxide, mesitylene, isophorone, methyl ethyl ketone, methyl propyl ketone, diacetone alcohol, 2,6-dimethylhepta-2,5-dien-4-one, and combinations thereof, wherein the weight percentages are based on the weight of the constituents in the distillate stream 105.

The higher boiling constituents in the liquid crude acetone feed stream 75 are removed as a bottoms product 84. The bottoms product 84 comprises greater than 98 weight % water, or greater than 99 weight % water, or greater than 99.5 weight % water, and further includes less than about 1 weight %, or less than about 0.5 weight %, or less than about 0.1 weight % of other impurities such as acetic acid, propionic acid, methyl isobutyl ketone, mesityl oxide, methyl ethyl ketone, isophorone, mesitylene, and other high boiling by-products of the ketonization reaction, wherein the weight % is based on the total weight of the constituents in the bottoms product stream 84.

A portion of the bottoms product stream 84 is vaporized by reboiler 120 to produce a vaporized bottoms stream 125 and returned to the distillation column 80 to provide heat and boil-up to the distillation column 80 contents.

Components that form azeotropes with water are at least a portion of the vaporized bottoms stream 125 and are withdrawn from the distillation column 80 via side draw stream 86. The portion of the column 80 above the side draw 86 and below the feed point for the distillation column 80 serves to strip acetone out of the side draw stream 86. Side draw stream 86 is fed to a decanter 130 and allowed to separate into two phases; an aqueous phase and an organics phase. At least a portion of the aqueous layer is returned to column 80 by line 135. The organic phase is withdrawn from the decanter 130 using line 140. Side draw stream 86 may be cooled to improve phase separation using any type of heat exchange mode known to those skilled in the art. The aqueous phase 135 returning to the column 80 may be heated to reduce the required heat load on reboiler 120. The composition of organic phase 140 comprises up to 80 weight percent or at least one acetic acid azeotrope-forming compound, about 1 to 10 weight percent water, and from 0.1 to 10 weight percent other organic species in small quantities in the liquid crude acetone feed, wherein the weight % is based on the total constituents of the organic phase 140. Generally, the side draw stream 86 has less than 5 weight percent acetone, or less than 2.5 weight percent acetone, or less than 1.5 weight percent acetone, based on the total weight of the constituents in the side draw stream 86.

Column 80 may be packed or trayed with any internals known in the art such as random, dumped, or structured packings, sieve, valve, bubble cap, and dual flow trays. Whether packed or trayed, the column may comprise 10 to 50 theoretical stages, or from 15 to 30 theoretical stages. The feed point for the liquid crude acetone 75 is typically from one-third to three-quarters of the stages from the bottom of the column, or can be from one-half to about five-eights of the stages from the bottom of the column 80. The location of the side draw is below the feed point and can be from at least two theoretical stages above the reboiler 120 to two theoretical stages below the feed point, or at least three theoretical stages above the reboiler 120 to four theoretical stages below the feed point.

Column 80 is operated at atmospheric pressure, with a head temperature about 56° C., bottoms temperature of about 100-104° C., and a temperature of the side draw tray of about 92-96° C. The column may also be operated at reduced pressure of from about 0.3 to about 1.0 bara pressure to reduce the reflux needed to achieve the desired acetone purity.

The reboiler 120 may be of any type known in the art, including but not limited to thermosyphon, kettle, or pot reboilers of vertical or horizontal design; wiped film, thin film, short path, or falling film evaporators.

The side draw typically consists of a total liquid draw off tray and liquid sump, with hat, bubble, or valve opens for vapor traffic.

The decanter may consist of any horizontal or vertical vessel with sufficient residence time to allow liquid-liquid disengagement and phase separation. Residence time is typically 5 minutes to eight hours, depending on the flow rate. The upper organic phase 140 is withdrawn at a rate to prevent accumulation of organics. The lower aqueous layer 135 is continuously returned to the column at a location within three theoretical stages of the side draw discharge point, or can be from one theoretical stage above or below the side draw discharge point. The lower layer may be heated prior to return to the column to reduce the reboiler 120 heat load.

Although the above side draw distillation process has been described in relation to ketonization of acetic acid, one skilled in the art will understand that the present invention is equally applicable to purification of acetone from more conventional methods utilized in the art. For example, one process for producing acetone includes dehydrogenation of 2-propanol. Propylene is absorbed in concentrated sulfuric acid to produce isopropyl sulfate, which is then hydrolyzed to 2-propanol. The 2-propanol is then oxidized to produce acetone.

Another method more commonly used is obtaining acetone as a co-product of phenol production where benzene is alkylated in the presence of a catalyst with propylene to produce cumene. Cumene is in turn oxidized to cumene hydroperoxide (CHP), which is then hydrolyzed in an acidic medium to yield phenol and acetone. Crude acetone resulting from the production of phenol from cumene typically contains about 200-700 ppm aldehydes and 200-500 ppm methanol.

The present invention is illustrated in greater detail by the specific examples presented below. It is to be understood that these examples are for illustrative purposes only and are not intended to be limiting of the invention, but rather are to be construed broadly within the scope and content of the appended claims. All parts and percentages in the examples are on a weight basis unless otherwise stated.

### Example 1

### Ketonization Reaction

A typical experimental ketonization procedure is as follows. Glacial acetic acid, comprising less than 0.5 weight percent of propionic acid, butyric acid, and other high boilers was mixed with water to produce a wet acid feed stream comprising 90.2 weight % acetic acid and 9.8 weight % water, based on the total weight of the sample. A 316 stainless steel tubular ketonization reactor, 1.5 inches inside diameter×24 inches long, with thermocouples situated every 2 inches in the reactor, was charged with 475.6 grams of a TiO₂ (anatase)/4% graphite (as binder) formed into 3/16 inch catalyst pellets. The depth of the catalyst bed was 12 inches. Quartz chips were loaded to a depth of 12 inches below and two inches above the catalyst bed. The reactor was wrapped with band insulation (about one-eighth inch thick), followed by electrical heat tap, and then covered in 6 inches of high temperature insulation. The reactor was connected, via one-half inch 316 stainless steel tubing, to an electrically heated tubing section acting as a vaporizer, which was connected, in turn, to an vaporizer unit comprising a 316 stainless steel 1.5 inches inside diameter×24 inches tube fitted with a 150-watt band heater, insulation, a dual-barrel syringe pump, and a level-controlled piston sludge pump. During operation of the reactor, the feed acid was pumped continuously to the vaporizer at 11.7 g/min. The temperature of the vaporizer was approximately 135° C. throughout the run. The feed acid was sludged out of the vaporizer at a rate averaging 19.9 weight % of the feed flow, based on the total weight of the feed. The acid sludge was found to contain about 94.1 weight % acetic acid, based on the total weight of the sludge. The vaporized wet acid stream, comprised about 89.1 weight % acetic acid with the remainder water, based on the weight of the wet acid stream, was then heated in the superheat section to about 440° C., and passed to the ketonization reactor at about 9.4 grams/minute flow rate. The ketonizaton reactor was operated in near-adiabatic mode (the heat tape added only sufficient heat to overcome non-reactive heat losses), with the average temperature of the catalyst bed about 420° C. The reactor effluent was condensed at 17° C., and allowed to collect in an overflow tank. The off gas from the tank was further contacted countercurrently with fresh water in an absorber. The absorber comprised an insulated 316 stainless steel tube, 8 feet long, 1 inch inside diameter filled with one-eighth inch 316 stainless steel Penn State packing. Fresh water was fed to the top of the absorber at a rate of 10 ml/min. A portion of the underflow from the absorber was passed through a water-cooled tube-in tube exchanger (temperature of 17° C.) and circulated at a rate of 36 ml/min to the center section of the column. The off gas from the absorber was passed through a dry ice trap to condense additional water and acetone. The volume of the off gas from the dry ice trap was measured by a flow meter and analyzed by gas chromatography. The remainder of the underflow stream, comprising acetone, water, and other heavy impurities, was combined with the overflow from the reactor condensation pot, and the dry ice trap liquids into a product tank. The contents of the product tank were weighed every 24 hours and analyzed by gas chromatography.

The ketonization reactor was operated continuously for 1128 hours, with a total feed to the vaporizer of 636 kilograms of acetic acid-water feed mixture. Conversion of acetic acid over the course of the run was 99.7%. A total of 875.7 kilograms of material was collected from the product tank over the course of the run, with an overall average composition given in Table 1. A total of 94,832 standard cubic liters of carbon dioxide off-gas was generated during operation, with the average composition shown in Table 2.

### Continuous Side Draw Distillation Column

A side draw distillation column, as laid out in FIG. 3, was used in Example 2 to distill crude acetone derived from ketonization of acetic acid. The distillation column comprised three silvered vacuum-jacketed glass column sections, 51 millimeters inside diameter, atop a metal reboiler, and a glass water-cooled condenser. The bottom section contained six Oldershaw trays. The middle section comprised a single total liquid draw-off hat tray, with a return port below the tray for re-introduction into the column of an aqueous layer from a side draw decanter. The top section of the column contained thirty-two Oldershaw trays.

Heat was supplied to the reboiler by electrical band heaters fitted to a Hastelloy vertical vessel, 51 millimeters inside diameter, 46 centimeters tall. The liquid level in the reboiler was measured by a calibrated pressure differential cell. The level was maintained at a set point (from 40 to 70% full) by linking the bottoms draw off pump to the liquid level measurement by an automatic control loop. The bottoms product was drawn off the reboiler as needed to maintain the desired liquid level using a variable speed piston. The hot bottoms product was conveyed to a chilled metal collection vessel, fitted with a glycol-cooled condenser. This pot was drained every 24 hours, with the contents weighed and thereafter analyzed by gas chromatography.

Vapors from the top of the column were conveyed using downward sloped glass tubing to a vertical glass water-cooled condenser. The condensed liquid drained into a 20 milliliter (ml) vertical collection vessel. Reflux to the column was pumped by a variable speed piston pump from the bottom of the collection vessel by a tubing to a port in the glass tubing at the top of the column. Reflux rate was set by the speed of the reflux pump. When the reflux pump rate was less than required to empty the condensate collection vessel at the given vapor flow rate from the column, then the condensate overflowed out of a side port on a collection vessel and drained by gravity to a distillate collection vessel. The distillate product drained into a chilled metal collection vessel, fitted with a glycol-cooled condenser. This pot was drained every 24 hours, with the contents weighed and analyzed using gas chromatography.

The side draw decanter consisted of a 500 ml glass vessel having an inlet port for liquid from the hat tray, a port for discharge of the upper organic phase, and a lower port for discharge of the bottom aqueous phase. Inlet flow to the decanter from the column was regulated by a manual needle valve. The bottom phase was connected by tubing to a pump and from the pump to the return port of the hat tray. The top organic layer was discharged by gravity drain through tubing by opening a valve periodically (typically once every few days) whenever the level buildup required reducing.

Feed to the column was supplied from a metal vessel containing the crude acetone. The metal feed tank was fitted with a glycol cooled condenser to ensure that acetone did not evaporate from the feed tank. This vessel was filled periodically with fresh crude acetone from the ketonization reactor/absorber system. Flow rate to the column was controlled by a variable speed piston pump. The feed was preheated to just below its boiling point by electrical heat tape wrapped around the metal tubing connecting the pump to the column.

### Description of Gas Chromatography Methods

Two GC methods were developed and employed to analyze samples:

GC Method-1 used a DB-Wax (60 meter×0.32 millimeter×1.0 um) capillary column and a thermal conductivity detector (TCD); samples were diluted in an internal standard solution that was injected onto the GC; this method provided weight percent composition of acetaldehyde, propionaldehyde, isobutyraldehyde, n-butyraldehyde, diethyl ether, acetone, water, isopropyl acetate, methyl ethyl ketone, isopropanol, isopropyl propionate, methyl propyl ketone, diethyl ketone, methyl isobutyl ketone, butyl acetate, mesityl oxide, dipropyl ketone, methyl amyl ketone, mesitylene, diacetone alcohol, isophorone.

Due to the limitation for accurate determination of organic acids by using the above direct injection GC Method-1, each sample was also analyzed by the following GC Method-2 that used a DB-1 (60 m×0.32 mm×1.0 um) capillary column and a flame ionization detector (FID); sample was first derivatized by reacting with BSTFA [N,O-bis(trimethylsilyl) trifluoroacetamide]; which converted the organic acids into their corresponding trimethylsilyl (TMS) esters. These esters are more volatile and inert for accurate quantification. Water can be accurately quantified as its bis-TMS derivative when sufficient BSTFA reagent was applied. This method provided accurate weight percent of acetic acid, propionic acid, isobutyric acid, butyric acid, and formic acid; This method can also be used to quantify weight percent alcohols (as TMS-ethers) and ketones (no derivatization reaction with BSTFA and detected as their original forms).

### Example 2

### Continuous Operation of Side Draw Column

This example illustrates the continuous distillation of crude acetone derived from the ketonization of acetic acid, followed by absorptive recovery in water, as described in Example 1 above. The side draw distillation column was as described above in Example 1. Results were analyzed by gas chromatography using methods previously described. The column was run continuously for eight days, with varying inlet crude acetone compositions as shown in Table 4. Distillate and bottoms product tanks were drained and weighed every eight hours, with the total for every 24 hour period combined together and analyzed once daily by gas chromatography. The side draw decanter product vessel was drained and weighed on days 3 and 8 and analyzed by gas chromatography. The overall weight collected on day 3 was assumed to be evenly distributed between days 1, 2, and 3. Similarly the side draw weight collected on day 8, was assumed to be evenly distributed between days 4 through 8. Column operational parameters such as feed, distillate, decanter and bottoms flows, temperatures, pressures, reflux ratio, and percent recovery of acetone, defined as, (acetone in distillate/acetone in feed), are summarized in Table 5. Compositions for the distillate, organic layer from the side draw decanter, and bottoms are given in Tables 5, 6, and 7 respectively. All measurements are in weight percentages, based on the total weight of the constituents, unless stated otherwise.

### Example 3

### Continuous Operation of Side Draw Column for Purification of Acetone Derived from Acetic Acid from Diketene Production

This example illustrates the continuous distillation of crude acetone derived from the acetic acid by-product from diketene production, followed by absorptive recovery in water. The side draw distillation column is as described above in Example 2. Results were analyzed by gas chromatography using methods previously described. The column was run continuously for two days. Average column operational parameters such as feed, distillate, decanter and bottoms flows, temperatures, distillate pressure, reflux ratio, percent recovery of acetone, defined as, (acetone in distillate/acetone in feed), and composition data (in weight percentages, based on the total weight of the constituents, unless stated otherwise) are summarized in Table 8. Not all components were speciated, as indicated by the designation “NS”. Some components were below the detection limit of the GC method as indicated by “ND”.

The following prophetic examples, Examples 4 through 8, illustrate the distillative purification of crude acetone derived from the ketonization of acetic acid from various sources. Impurities are removed using the side draw decanter organic stream as illustrated in FIG. 3. In each example, the simulation modeled a column with 20 theoretical stages. The feed point was on stage 12 from the bottom, and the side draw off was from stage 4 from the bottom. The water layer was returned on stage three from the bottom. The reflux ratio, approximately 0.75 to 0.95 and boilup were manipulated in the simulation to give 99.9% recovery of acetone with 2 weight % water in the distillate. The side draw purge rate was set to ensure greater than 99.9% removal of combined impurities such as isophorone, mesitylene, mesityl oxide and other case-specific impurities that form azeotropes with water and not with acetone such as limonene, pinene, isobutylbenzene, DIPK, isopropyl isopropenyl ketone, isopropenyl acetate, 2,4-pentanedione, and the like. Stream numbers correspond to those of FIG. 3.

The crude acetone feed to the side draw distillation was derived from acetic acid containing impurities associated with each of the following processes:

Example 4—purified acetic acid;

Example 5—the acetylation of wood;

Example 6—the preparation of a specialty chemical by acetylation;

Example 7—the preparation of isobutyric anhydride by the acetylation of isobutyric acid with acetic anhydride derived from the process for the preparation of TMCD; and

Example 8—the production of diketene

### Example 4

### Simulation of Side Draw Column for Purification of Acetone Derived from Purified Acetic Acid

The following prophetic example illustrates the distillative purification, by the side draw column configuration described above, of crude acetone prepared by the ketonization of purified acetic acid. Material balance results from the simulation of said distillation are given in Table 9.

### Example 5

### Simulation of Side Draw Column for Purification of Acetone Derived from Acetic Acid from Wood Acetylation

The following prophetic example illustrates the distillative purification, by the side draw column configuration described above, of crude acetone prepared by the ketonization of acetic acid derived from wood acetylation. Material balance results from the simulation of said distillation are given in Table 10.

### Example 6

### Simulation of Side Draw Column for Purification of Acetone Derived from Acetic Acid from Specialty Chemical Acetylation

The following prophetic example illustrates the distillative purification, by the side draw column configuration described above, of crude acetone prepared by the ketonization of acetic acid derived from specialty chemical acetylation. Material balance results from the simulation of said distillation are given in Table 11.

### Example 7

### Simulation of Side Draw Column for Purification of Acetone Derived from Acetic Acid from Isobutyric Anhydride Production

The following prophetic example illustrates the distillative purification, by the side draw column configuration described above, of crude acetone prepared by the ketonization of acetic acid derived from isobutyric anhydride used in TMCD preparation. Material balance results from the simulation of said distillation are given in Table 12.

### Example 8

### Simulation of Side Draw Column for Purification of Acetone Derived from Acetic Acid from Diketene Production

The following prophetic example illustrates the distillative purification, by the side draw column configuration described above, of crude acetone prepared by the ketonization of acetic acid derived from diketene production. Material balance results from the simulation of said distillation are given in Table 13.

Material balance results from the simulation of said distillation are given in Table 8.

TABLE 1
Condensed Reactor Effluent Composition, Weight %

| | |
|---|---|
| Acetone | 32.76 |
| Acetic Acid | 0.22 |
| Water | 66.72 |
| Mesityl Oxide | 0.25 |
| Mesitylene | 0.0002 |
| Isophorone | 0.02 |
| Methyl ethyl Ketone | 0.0015 |
| Others | 0.044 |

TABLE 2
Reactor Off-Gas Composition, Mole %

| | |
|---|---|
| Hydrogen | 0.06 |
| Carbon dioxide | 98.53 |
| Methane | 0.10 |
| Acetone | 0.04 |
| isobutylene | 0.11 |
| Water | 1.15 |
| MEK and other by-products | 0.01 |

TABLE 3
Feed to Distillation Zone 6, Crude Acetone Composition, Weight %

| Component | Day 1 | Day 2 | Day 3 | Day 4 | Day 5 | Day 6 | Day 7 | Day 8 | Total |
|---|---|---|---|---|---|---|---|---|---|
| Acetone | 23.53 | 36.14 | 39.20 | 34.47 | 36.73 | 37.80 | 49.16 | 48.41 | 37.08 |
| MEK | 0.00 | 0.00 | 0.00 | 0.02 | 0.02 | 0.01 | 0.02 | 0.02 | 0.009 |
| MIBK | 0.00 | 0.00 | 0.00 | 0.00 | 0.02 | 0.00 | 0.00 | 0.00 | 0.00 |
| Water | 76.18 | 63.63 | 60.22 | 65.10 | 62.83 | 61.14 | 50.54 | 50.87 | 62.42 |
| Mesityl Oxide | 0.05 | 0.03 | 0.15 | 0.10 | 0.08 | 0.07 | 0.03 | 0.42 | 0.10 |
| Mesitylene | 0.00 | 0.00 | 0.00 | 0.00 | 0.00 | 0.00 | 0.00 | 0.01 | 0.0014 |
| Diacetone Alcohol | 0.00 | 0.00 | 0.00 | 0.00 | 0.00 | 0.00 | 0.00 | 0.00 | 0.0002 |
| Isophorone | 0.00 | 0.00 | 0.00 | 0.01 | 0.01 | 0.00 | 0.00 | 0.02 | 0.0045 |
| Acetic Acid | 0.08 | 0.07 | 0.07 | 0.28 | 0.27 | 0.26 | 0.21 | 0.21 | 0.18 |
| Unknowns | 0.15 | 0.13 | 0.37 | 0.03 | 0.05 | 0.71 | 0.04 | 0.05 | 0.20 |

TABLE 4
Side Draw Column Parameters

| Parameter | Day 1 | Day 2 | Day 3 | Day 4 | Day 5 | Day 6 | Day 7 | Day 8 | Avg. |
|---|---|---|---|---|---|---|---|---|---|
| Feed Rate, g/min | 21.4 | 20.3 | 20.2 | 21.1 | 20.3 | 20.7 | 14.2 | 12.6 | 18.9 |
| Dist Rate, g/min | 5.1 | 7.5 | 8.2 | 7.5 | 7.7 | 8.2 | 7.1 | 6.4 | 7.2 |
| Decant Org flow, g/min | 0.006 | 0.007 | 0.009 | 0.018 | 0.017 | 0.010 | 0.005 | 0.039 | 0.014 |
| Bottoms Rate, g/min | 16.3 | 12.8 | 12.0 | 13.6 | 12.7 | 12.5 | 7.0 | 6.2 | 11.6 |
| Reflux Ratio | 1.28 | 0.88 | 0.80 | 0.88 | 0.86 | 0.80 | 0.90 | 1.06 | |
| Dist temp (° C.) (Stage 40) | 57.1 | 57.1 | 57.5 | 57.2 | 58.0 | 57.7 | 57.7 | 58.5 | |
| Side Draw temp (° C.) (Stage 7) | 93.8 | 95.4 | 95.1 | 96.0 | 96.3 | 96.0 | 95.9 | 95.1 | |
| Bottoms temp (° C.) (Stage 1) | 101.6 | 101.5 | 101.4 | 101.6 | 101.6 | 101.6 | 101.4 | 101.3 | |
| Column Head Press (bara) | 0.98 | 0.98 | 0.99 | 0.98 | 0.99 | 0.99 | 0.99 | 0.99 | |
| Acetone Recovery, % | 99.8 | 99.9 | 99.9 | 100.0 | 100.0 | 100.0 | 100.0 | 100.0 | 100.0 |

TABLE 5
Distillate Compositions, Weight %

| Component | Day 1 | Day 2 | Day 3 | Day 4 | Day 5 | Day 6 | Day 7 | Day 8 | Avg. |
|---|---|---|---|---|---|---|---|---|---|
| Acetone | 97.95 | 98.13 | 96.62 | 97.41 | 97.40 | 95.16 | 97.36 | 95.71 | 96.92 |
| MEK | 0.00 | 0.00 | 0.00 | 0.05 | 0.04 | 0.03 | 0.03 | 0.04 | 0.02 |
| MIBK | 0.00 | 0.00 | 0.00 | 0.00 | 0.05 | 0.00 | 0.00 | 0.00 | 0.01 |
| Water | 1.92 | 1.84 | 2.47 | 2.46 | 2.40 | 3.01 | 2.58 | 3.89 | 2.58 |
| Mesityl Oxide | 0.09 | 0.00 | 0.27 | 0.08 | 0.04 | 0.06 | 0.00 | 0.32 | 0.11 |
| Mesitylene | 0.00 | 0.00 | 0.00 | 0.00 | 0.00 | 0.00 | 0.00 | 0.00 | 0.00 |
| Di-Acetone Alcohol | 0.00 | 0.00 | 0.00 | 0.00 | 0.00 | 0.00 | 0.00 | 0.00 | 0.00 |
| Isophorone | 0.00 | 0.00 | 0.00 | 0.00 | 0.00 | 0.00 | 0.00 | 0.00 | 0.00 |
| Acetic Acid | 0.00 | 0.00 | 0.01 | 0.00 | 0.00 | 0.00 | 0.00 | 0.00 | 0.00 |
| Unknowns | 0.03 | 0.03 | 0.62 | 0.00 | 0.07 | 1.73 | 0.03 | 0.04 | 0.36 |

TABLE 6
Decanter Organic Layer Compositions, Weight Percent

| Component | Day 1 | Day 2 | Day 3 | Day 4 | Day 5 | Day 6 | Day 7 | Day 8 | Total |
|---|---|---|---|---|---|---|---|---|---|
| Acetone | 0.05 | 0.05 | 0.05 | 0.00 | 0.00 | 0.00 | 0.00 | 0.00 | 0.01 |
| MEK | 0.00 | 0.00 | 0.00 | 0.00 | 0.00 | 0.00 | 0.00 | 0.00 | 0.00 |
| MIBK | 0.03 | 0.03 | 0.03 | 0.07 | 0.07 | 0.07 | 0.07 | 0.07 | 0.07 |
| Water | 3.67 | 3.67 | 3.67 | 5.14 | 5.14 | 5.14 | 5.14 | 5.14 | 4.84 |
| Mesityl Oxide | 80.85 | 80.85 | 80.85 | 82.29 | 82.29 | 82.29 | 82.29 | 82.29 | 82.00 |
| Mesitylene | 1.05 | 1.05 | 1.05 | 2.05 | 2.05 | 2.05 | 2.05 | 2.05 | 1.85 |
| Di-Acetone Alcohol | 0.78 | 0.78 | 0.78 | 0.21 | 0.21 | 0.21 | 0.21 | 0.21 | 0.33 |
| Isophorone | 3.71 | 3.71 | 3.71 | 6.76 | 6.76 | 6.76 | 6.76 | 6.76 | 6.15 |
| Acetic Acid | 0.07 | 0.07 | 0.07 | 0.29 | 0.29 | 0.29 | 0.29 | 0.29 | 0.25 |
| Unknowns | 9.80 | 9.80 | 9.80 | 3.18 | 3.18 | 3.18 | 3.18 | 3.18 | 4.51 |

TABLE 7
Bottoms Product Compositions, Weight Percent

| Component | Day 1 | Day 2 | Day 3 | Day 4 | Day 5 | Day 6 | Day 7 | Day 8 | Total |
|---|---|---|---|---|---|---|---|---|---|
| Acetone | 0.05 | 0.05 | 0.05 | 0.00 | 0.00 | 0.00 | 0.00 | 0.00 | 0.02 |
| MEK | 0.00 | 0.00 | 0.00 | 0.00 | 0.00 | 0.00 | 0.00 | 0.00 | 0.00 |
| MIBK | 0.00 | 0.00 | 0.00 | 0.00 | 0.00 | 0.00 | 0.00 | 0.00 | 0.00 |
| Water | 99.66 | 99.66 | 99.66 | 99.53 | 99.53 | 99.53 | 99.53 | 99.53 | 99.59 |
| Mesityl Oxide | 0.00 | 0.00 | 0.00 | 0.00 | 0.00 | 0.00 | 0.00 | 0.00 | 0.00 |
| Mesitylene | 0.00 | 0.00 | 0.00 | 0.00 | 0.00 | 0.00 | 0.00 | 0.00 | 0.00 |
| Di-Acetone Alcohol | 0.00 | 0.00 | 0.00 | 0.00 | 0.00 | 0.00 | 0.00 | 0.00 | 0.00 |
| Isophorone | 0.00 | 0.00 | 0.00 | 0.00 | 0.00 | 0.00 | 0.00 | 0.00 | 0.00 |
| Acetic Acid | 0.11 | 0.11 | 0.11 | 0.43 | 0.43 | 0.43 | 0.43 | 0.43 | 0.29 |
| Unknowns | 0.18 | 0.18 | 0.18 | 0.04 | 0.04 | 0.04 | 0.04 | 0.04 | 0.10 |

TABLE 8
Side Draw Distillation of Acetone Derived from Diketene Production Side Draw
Distillation of Acetone Derived from Acetic Acid from Diketene Production

| | Column | | | | Organic |
|---|---|---|---|---|---|
| | Parameters | Feed | Distillate | Bottoms | Side Draw |
| Mass Flow Rate(g/min) | | 15.70 | 8.21 | 7.10 | 0.39 |
| Temperature (° C.) | | 22 | 57.7 | 101.6 | 96 |
| Column Head Pressure (bara) | 0.98 to 0.99 | | | | |
| Average Reflux Ratio | 0.8 to 0.93 | | | | |
| Acetone Recovery (%) | 98.5% | | | | |

| | |
|---|---|
| Component | Weight Percentage |

| | | | | |
|---|---|---|---|---|
| Acetic Acid | 0.58% | 0.00% | 1.90% | 0.00% |
| Water | 44.61% | 0.40% | 97.89% | 5.20% |
| Acetone | 52.91% | 99.60% | 0.00% | 32.89% |
| Mesityl Oxide | 0.58% | 5.5 ppm | 0.00% | 25.15% |
| Isophorone | 0.08% | ND | 0.00% | 1.93% |
| Others* | 1.24% | ND | 0.00% | 34.82% |
| unknowns | NS | 77 ppm | ND | NS |

*includes diketene, 2,4-pentanedione, isopropenyl acetate, 2,4,6-heptatrione, 2,6-dimethylpyrone, and dehydroacetic acid

TABLE 9
Simulation Results for Side Draw Distillation of Acetone
Derived from Ketonization of Purified Acetic Acid

| | Feed | Underflow | Vent | Distillate | |
|---|---|---|---|---|---|
| | Stream 75 | Steam 84 | Stream 95 | Stream 105 | Organic Layer Stream 140 |
| Temperature (° C.) | 64.8 | 107.4 | 31 | 31 | 44.45 |
| Pressure (bara) | 2.39 | 1.31 | 1.03 | 1.03 | 1.21 |
| Mass flow rate (kg/hr) | 1000.6 | 678.4 | 0.2 | 317.5 | 4.6 |
| Component | Mass flow rate (lb/hr) | | | | |
| CO₂ | 2.68 | <0.0001 | 0.098 | 2.6 | <0.0001 |
| Isobutylene | <0.0001 | <0.0001 | <0.0001 | <0.0001 | <0.0001 |
| Acetone | 308.2 | <0.0001 | 0.076 | 308.1 | <0.0001 |
| Water | 684.2 | 677.8 | <0.0001 | 6.35 | <0.0001 |
| Acetic Acid | 0.27 | 0.27 | <0.0001 | <0.0001 | <0.0001 |
| Mesityl oxide | 4.55 | 0.26 | <0.0001 | 0.0007 | 4.29 |
| Mesitylene | 0.00 | 0.00 | <0.0001 | 0.0012 | 0.00 |
| Isophorone | 0.09 | 0.00 | <0.0001 | 0.0000 | 0.09 |

TABLE 10
Simulation Results for Side Draw Distillation of Acetone
Derived from Acetic Acid from Wood Acetylation

| | Feed | Underflow | Vent | Distillate | Organic Layer |
|---|---|---|---|---|---|
| | Stream 75 | Steam 84 | Stream 95 | Stream 105 | Stream 140 |
| Temperature (° C.) | 64.8 | 107.4 | 31 | 31 | 44.45 |
| Pressure (bara) | 2.39 | 1.31 | 1.03 | 1.03 | 1.21 |
| Mass flow rate (kg/hr) | 1000.0 | 678.0 | 0.2 | 317.3 | 4.6 |
| Component | Mass flow rate (lb/hr): | | | | |
| CO₂ | 2.66 | <0.0001 | 0.097 | 2.6 | <0.0001 |
| Isobutylene | 0.00 | <0.0001 | 0.000 | 0.0 | <0.0001 |
| Acetone | 305.92 | 0.18 | 0.076 | 305.6 | 0.03 |
| Water | 679.21 | 672.86 | 0.001 | 6.3 | 0.05 |
| Acetic Acid | 0.27 | 0.27 | 0.00000 | 0.000 | 0.00 |
| Mesityl oxide | 4.52 | 0.06 | 0.00000 | 0.001 | 4.46 |
| Mesitylene | 0.00 | 0.00000 | 0.00000 | 0.001 | 0.001 |
| Isophorone | 0.08 | 0.00000 | 0.00000 | 0.000 | 0.08 |
| Limonene | 6.12 | 0.00000 | 0.00000 | 0.001 | 6.12 |
| Alpha Pinene | 1.22 | 0.00000 | 0.00000 | 0.001 | 1.22 |

TABLE 11
Simulation Results for Side Draw Distillation of Acetone
Derived from Acetic Acid from Specialty Chemical Acetylation

| | Feed | Underflow | Vent Stream | Distillate | Organic Layer |
|---|---|---|---|---|---|
| | Stream 75 | Steam 84 | 95 | Stream 105 | Stream 140 |
| Temperature (° C.) | 64.8 | 107.4 | 31 | 31 | 44.45 |
| Pressure (bara) | 2.39 | 1.31 | 1.03 | 1.03 | 1.21 |
| Mass flow rate (kg/hr) | 1000.0 | 678.0 | 0.2 | 317.3 | 4.6 |
| Component | Mass flow rate (lb/hr) | | | | |
| CO₂ | 2.62 | 0.00 | 0.096 | 2.5 | <0.0001 |
| Isobutylene | 0.00 | 0.00 | 0.000 | 0.0 | <0.0001 |
| Acetone | 301.76 | 0.17 | 0.075 | 301.5 | 0.03 |
| Water | 669.99 | 663.73 | 0.001 | 6.2 | 0.05 |
| Acetic Acid | 0.26 | 0.26 | 0.00000 | 0.000 | 0.00 |
| Mesityl oxide | 4.46 | 0.06 | 0.00000 | 0.001 | 4.40 |
| Mesitylene | 0.00 | 0.00 | 0.00000 | 0.001 | 0.001 |
| Isophorone | 0.08 | 0.00 | 0.00000 | 0.000 | 0.08 |
| Isobutylbenzene | 15.99 | 0.00 | 0.00000 | 0.002 | 15.99 |
| Isobutyl Acetophenone | 4.83 | 4.82 | 0.00000 | 0.005 | 0.00 |
| Hydrogen Fluoride | 0.02 | 0.02 | 0.00000 | 0.000 | 0.00 |

TABLE 12
Simulation Results for Side Draw Distillation of
Acetone Derived from Isobutyric Anhydride Production

| | Feed | Underflow | Vent | Distillate | Organic Layer |
|---|---|---|---|---|---|
| | Stream 75 | Steam 84 | Stream 95 | Stream 105 | Stream 140 |
| Temperature (° C.) | 64.8 | 107.4 | 31 | 31 | 44.45 |
| Pressure (bara) | 2.39 | 1.31 | 1.03 | 1.03 | 1.21 |
| Mass flow rate (kg/hr) | 1000.0 | 678.0 | 0.2 | 317.3 | 4.6 |
| Component | Mass flow rate (lb/hr) | | | | |
| CO₂ | 2.67 | 0.00 | 0.098 | 2.6 | <0.0001 |
| Isobutylene | 0.00 | 0.00 | 0.000 | 0.0 | <0.0001 |
| Acetone | 307.94 | 0.18 | 0.076 | 307.7 | 0.03 |
| Water | 683.71 | 677.31 | 0.001 | 6.3 | 0.05 |
| Acetic Acid | 0.27 | 0.27 | 0.00000 | 0.000 | 0.00 |
| Mesityl oxide | 4.55 | 0.06 | 0.00000 | 0.001 | 4.49 |
| Mesitylene | 0.00 | 0.00 | 0.00000 | 0.001 | 0.001 |
| Isophorone | 0.09 | 0.00 | 0.00000 | 0.000 | 0.09 |
| Tetramethylethylene | 0.43 | 0.00 | 0.00043 | 0.388 | 0.04 |
| 2,4-Dimethyl-1,3 pentadiene | 0.34 | 0.00 | 0.00000 | 0.000 | 0.34 |
| DIPK | 1.72 | 0.00 | 0.00000 | 0.002 | 1.72 |
| Isopropyl isopropenyl ketone | 5.74 | 0.02 | 0.00000 | 0.006 | 5.71 |

TABLE 13
Simulation Results for Side Draw Distillation of
Acetone Derived from Diketene Production

| | Feed | Underflow | Vent | Distillate | Organic Layer |
|---|---|---|---|---|---|
| | Stream 75 | Steam 84 | Stream 95 | Stream 105 | Stream 140 |
| Temperature (° C.) | 64.8 | 107.4 | 31 | 31 | 44.45 |
| Pressure (bara) | 2.39 | 1.31 | 1.03 | 1.03 | 1.21 |
| Mass flow rate (kg/hr) | 1000.0 | 678.0 | 0.2 | 317.3 | 4.6 |
| Component | Mass flow rate (lb/hr) | | | | |
| CO₂ | 2.66 | <0.0001 | 0.097 | 2.6 | <0.0001 |
| Isobutylene | 0.00 | <0.0001 | 0.000 | 0.0 | <0.0001 |
| Acetone | 306.57 | 0.18 | 0.076 | 306.3 | 0.03 |
| Water | 680.67 | 674.31 | 0.001 | 6.3 | 0.05 |
| Acetic Acid | 0.27 | 0.27 | 0.00000 | 0.000 | 0.00 |
| Mesityl oxide | 4.53 | 0.06 | 0.00000 | 0.001 | 4.47 |
| Mesitylene | 0.00 | 0.00000 | 0.00000 | 0.001 | 0.001 |
| Isophorone | 0.09 | 0.00000 | 0.00000 | 0.000 | 0.08 |
| 2,4-pentanedione | 3.99 | 0.00000 | 0.00000 | 0.001 | 3.98 |
| Isopropenyl acetate | 1.23 | 0.00000 | 0.00000 | 0.010 | 1.22 |

Having described the invention in detail, those skilled in the art will appreciate that modifications may be made to the various examples of the invention without departing from the scope and spirit of the invention disclosed and described herein. It is, therefore, not intended that the scope of the invention be limited to the specific examples illustrated and described but rather it is intended that the scope of the present invention be determined by the appended claims and their equivalents.

We claim:

1. A process for the purification of acetone, comprising:
(a) feeding a crude product mixture comprising acetone, water, and an impurity to a distillation column;
(b) withdrawing a liquid side draw stream from the distillation column;
(c) separating the side draw stream into a water layer and an organic layer, and returning the water layer to the distillation column; and
(d) recovering from the distillation column: (i) a lower boiling fraction comprising a purified acetone and (ii) a higher boiling fraction comprising a major amount of the water.

2. The process of claim 1 wherein said crude product mixture is an acetic acid ketonization product stream from a ketonization reaction comprising acetone, water, an impurity and ketonization reaction by-products.

3. The process of claim 1 wherein said impurity comprises at least one acetic acid azeotrope-forming impurity selected from the group consisting of an alkyl aromatic hydrocarbon, an aldehyde, a ketone, an aromatic ester, an acyclic ester, a terpene, a terpenoid, an acyclic unsaturated hydrocarbon, and combinations thereof.

4. The process according to claim 3, wherein the crude product mixture comprises about 25 to about 70 weight percent acetone, about 25 to about 75 weight percent water, and about 10 ppm to about 25 weight percent of said impurity, based on the total weight of the crude product mixture.

5. The process according to claim 2, wherein the ketonization by-products comprise mesityl oxide, mesitylene, isophorone, methyl ethyl ketone, methyl propyl ketone, diacetone alcohol, 2,6-dimethylhepta-2,5-dien-4-one, and combinations thereof.

6. The process according to claim 2, wherein the ketonization product stream is prepared by contacting a vaporized acetyl feed stream comprising acetic acid with at least one metal oxide catalyst selected from the group consisting of titanium, zirconium, thorium, cerium, lanthanum, magnesium, aluminum, and mixtures thereof and at a temperature of from about 350° C. to about 650° C. and a pressure of from about 10 kPa to about 3000 kPa in a ketonization reactor.

7. The process according to claim 6 wherein said metal oxide catalyst further comprises from about 1 weight percent to about 25 weight percent, based on the total weight of the catalyst, of lithium, sodium, potassium, cesium, or mixtures thereof.

8. The process according to claim 6, wherein the vaporized acetyl feed stream comprises acetic acid recovered from a process selected from the group consisting of wood acetylation; acetylation of alcohols with acetic anhydride to form esters; carbonylation of methanol and methyl acetate to form acetic acid and acetic anhydride; preparation of 2,2,4,4-tetramethyl-1,3-cyclobutanediol from isobutyric anhydride via acetic anhydride exchange; preparation of ketenes and diketene from acetic acid; condensation of phenyl acetate monomers; preparation of fine chemicals and pharmaceuticals; preparation of carboxylic acid anhydrides from their corresponding acids by exchange with acetic anhydride; acylation reactions and mixtures thereof.

9. The process of claim 6 wherein said acetyl feed stream comprises about 40 to about 99 weight percent acetic acid, up to about 50 weight of the impurity, and optionally up to about 30 weight percent water, wherein the weight % is based on the total weight of the feed stream.

10. The process according to claim 2, wherein the liquid side draw stream comprises up to 5 weight percent acetone, based on the total weight of the liquid side draw stream, and the organic layer comprises about 40 to about 90 weight percent mesityl oxide, isophorone, mesitylene and mixtures thereof, and about 0.01 to about 0.5 weight percent acetic acid, based on the weight of the organic layer.

11. The process of claim 2 wherein said lower boiling fraction comprises about 95 to about 99 weight percent acetone, about 0.1 to about 5 weight percent water and from about 50 ppm to about 1.0 weight percent of the impurity, based on the total weight of the lower boiling fraction; and the higher boiling fraction comprising greater than 50 weight percent water, and about 1 weight % of the impurity and ketonization byproducts, based on the total weight of the higher boiling fraction.

12. The process of claim 11 wherein said higher boiling fraction comprises greater than about 90 weight % water and from about 0.005 to about 1 weight % of the impurity and the ketonization byproducts.

13. The process according to claim 11, wherein the higher boiling fraction comprises greater than about 99.5 weight percent water, based on the total weight of the higher boiling fraction.

14. A process for preparing a purified ketone from an acetic acid containing stream comprising the steps of:
a) contacting the acetic acid containing feed stream comprising acetic acid and an impurity comprising at least one acetic acid azeotrope-forming compound with a meal oxide catalyst in a ketonization reactor to produce a crude product mixture by a ketonization reaction wherein the crude product mixture comprises acetone, water, the impurity, and by-products from the ketonization reaction;
b) feeding the crude product mixture comprising acetone, water, and an impurity to a distillation column;
c) withdrawing a liquid side draw stream from the distillation column;
d) separating the side draw stream into a water layer and an organic layer, and returning the water layer to the distillation column; and
e) recovering from the distillation column: (i) a lower boiling fraction comprising a purified acetone relative to the crude product mixture and (ii) a higher boiling fraction comprising a major amount of the water.

15. The process according to claim 14, wherein the acetic acid ketonization product stream is prepared from an acetic acid feed recovered from a process selected from the group consisting of wood acetylation; acetylation of alcohols with acetic anhydride to form esters; carbonylation of methanol and methyl acetate to form acetic acid and acetic anhydride; preparation of 2,2,4,4-tetramethyl-1,3-cyclobutanediol from isobutyric anhydride via acetic anhydride exchange; preparation of ketenes and diketene from acetic acid; condensation of phenyl acetate monomers; preparation of fine chemicals and pharmaceuticals; preparation of carboxylic acid anhydrides from their corresponding acids by exchange with acetic anhydride; acylation reactions and mixtures thereof.

16. The process according to claim 14 wherein the acetyl feed stream comprises about 40 to about 99 weight percent acetic acid, up to about 50 weight of the impurity, and optionally up to about 30 weight percent water.

17. The process according to claim 14 wherein the crude product mixture comprises about 25 to about 70 weight percent acetone, about 25 to about 75 weight percent water, and about 0.05 weight % to about 25 weight percent of said impurity.

18. The process of claim 14 wherein said recovering non-condensable components comprises contacting said crude product mixture with an absorbent in a countercurrent absorber to produce the liquid crude acetone stream.

19. The process according to claim 18 wherein the absorbent comprises water.

20. The process according to claim 14 wherein the liquid side draw stream comprises up to 5 weight percent acetone, based on the total weight of the liquid side draw stream, and the organic layer comprises about 40 to about 90 weight percent mesityl oxide, isophorone, and mixtures thereof, and about 0.01 to about 0.5 weight percent acetic acid, based on the weight of the organic layer.

21. The process of claim 14 wherein said lower boiling fraction comprises about 95 to about 99 weight percent acetone, about 0.1 to about 5 weight percent water and from about 50 ppm to about 1.0 weight percent of the impurity, based on the weight of the lower boiling fraction; and the higher boiling fraction comprising greater than 50 weight percent water, and about 1 weight % of the impurity and the ketonization byproducts, based on the total weight of the higher boiling fraction.

22. The process of claim 21 wherein said higher boiling fraction comprises greater than about 90 weight % water and from about 0.005 to about 1 weight % of the impurity and the ketonization byproducts, based on the total weight of the higher boiling fraction.

23. The process according to claim 21, wherein the higher boiling fraction comprises about 99.5 weight percent water, based on the total weight of the higher boiling fraction.

24. A process for preparing a purified ketone from an acetic acid containing stream comprising the steps of:
a) vaporizing an acetyl feed stream comprising acetic acid, an impurity comprising at least one acetic acid azeotrope-forming compound, and 0-50 weight % water, optionally mixing steam with the vaporized acetyl feed stream to produce a vaporized feed mixture;
b) superheating the vaporized feed mixture to produce a superheated feed mixture;
c) contacting the superheated feed mixture with a metal oxide catalyst in a ketonization reactor to produce a crude product mixture comprising acetone, water, the impurity, carbon dioxide, and byproducts from the ketonization reaction;
d) recovering condensable components of the crude product mixture to produce a recovered liquid crude acetone stream comprising acetone, water, an impurity and ketonization by-products and a gaseous off-gas stream;
e) feeding the recovered liquid crude acetone stream to a distillation column;
f) withdrawing a liquid side draw stream from the distillation column;
g) separating the side draw stream into a water layer and an organic layer, and returning the water layer to the distillation column; and
h) recovering from the distillation column: (i) a lower boiling fraction comprising a purified acetone relative to the crude product mixture and (ii) a higher boiling fraction comprising a major amount of the water.

25. The process according to claim 24 wherein the acetyl feed stream comprises about 40 to about 99 weight percent acetic acid, up to about 50 weight of the impurity, and optionally up to about 30 weight percent water.

26. The process according to claim 24 wherein the crude product mixture comprises about 25 to about 70 weight percent acetone, about 25 to about 75 weight percent water, and about 0.05 weight % to about 25 weight percent of said impurity.

27. The process according to claim 24 wherein the liquid side draw stream comprises up to 5 weight percent acetone, based on the total weight of the liquid side draw stream, and the organic layer comprises about 40 to about 90 weight percent mesityl oxide, isophorone, and mixtures thereof, and about 0.01 to about 0.5 weight percent acetic acid, based on the weight of the organic layer.

28. The process of claim 24 wherein said lower boiling fraction comprises about 95 to about 99 weight percent acetone, about 0.1 to about 5 weight percent water and from about 50 ppm to about 1.0 weight percent of the impurity, based on the weight of the lower boiling fraction; and the higher boiling fraction comprising greater than 50 weight percent water, and about 1 weight % of the impurity and the ketonization byproducts, based on the total weight of the higher boiling fraction.

29. The process according to claim 24, wherein the higher boiling fraction comprises greater than about 99.5 weight percent water, based on the total weight of the higher boiling fraction.
